(19) United States
(12) Patent Application Publication
(10) Pub. No.: US 2008/0199527 A1
(43) Pub. Date: Aug. 21, 2008

(54) Enteric Coated Azithromycin Multiparticulates

(75) Inventors: William J. Curatolo, Niantic, CT (US); Scott M. Herbig, East Lyme, CT (US); Steven R. LeMott, East Lyme, CT (US); Julian B. Lo, Old Lyme, CT (US); Leah E. Appel, Bend, OR (US); Dwayne T. Friesen, Bend, OR (US); David K. Lyon, Bend, OR (US); Scott B. McCray, Bend, OR (US); James B. West, Miramar, FL (US)

Correspondence Address: PFIZER INC., PATENT DEPARTMENT, MS8260-1611, EASTERN POINT ROAD, GROTON, CT, 06340, US

(73) Assignee: Pfizer Inc., Groton, CT (US)

(21) Appl. No.: 11/722,364

(22) PCT Filed: Dec. 9, 2005
(86) PCT No.: PCT/IB05/03764

§ 371 Date: Aug. 1, 2007

Related U.S. Application Data
(60) Provisional application No. 60/638,287, filed on Dec. 21, 2004.

(51) Int. Cl. A61K 9/14; A61K 31/7048

(52) U.S. Cl. 424/494; 424/489; 514/29; 424/497; 424/498; 424/499

(57) ABSTRACT

A pharmaceutical composition is disclosed which comprises multiparticulates wherein said multiparticulates further comprise an azithromycin core and an enteric coating disposed upon said azithromycin core.

### BACKGROUND OF THE INVENTION

Azithromycin is an antibiotic which is administered orally or intravenously, to treat various infections, particularly infections of the urinary tract, bronchial tract, lungs, sinuses and the middle ear.

Oral dosing of azithromycin can result in adverse gastrointestinal (GI) side effects such as nausea, cramping, diarrhea and vomiting in a significant number of patients.

The frequency of these adverse effects increase with higher dose levels of azithromycin. In treating adult humans, for a single 1 gram dose, administered in an oral suspension, the reported incidence of various GI side effects was 7% diarrhea/loose stools, 5% nausea, 5% abdominal pain, and 2% vomiting (U.S. Package Insert for Zithromax® azithromycin for oral suspension). However, for a single 2 gram, administered in an oral suspension, the reported incidence of various GI side effects was 14% diarrhea/loose stools, 7% abdominal pain, and 7% vomiting (Ibid.).

Therefore, what is needed is an azithromycin dosage form that has a bioavailability similar to, and gastrointestinal side effects less than, an equivalent dose of immediate release azithromycin.

### SUMMARY OF THE INVENTION

The present invention relates to a pharmaceutical composition comprising multiparticulates wherein said multiparticulates further comprise an azithromycin core and an enteric coating disposed upon said core.

The pharmaceutical composition of the present invention provides an enterically coated multiparticulate controlled release azithromycin-dosage form that decreases, relative to currently available immediate release azithromycin dosage forms that deliver an equivalent dose, the incidence and/or severity of GI side effects.

### DETAILED DESCRIPTION OF THE INVENTION

As used in the present invention, the term “about” means the specified value ±10% of the specified value.

As used in the present invention, the terms “a” or “an” mean one or more. For example, the term “an alkalizing agent” means one or more alkalizing agents, the term “a carrier” means one or more carriers, and the term “a dissolution enhancer” means one or more dissolution enhancers.

The term “pharmaceutically acceptable”, as used herein, means that which is compatible with other ingredients of the composition, and not deleterious to the recipient thereof.

The term “multiparticulate” as used herein is intended to embrace a dosage form comprising a multiplicity of coated particles whose totality represents the intended therapeutically useful dose of azithromycin. The particles generally have a mean diameter from about 10 μm to about 3000 μm, preferably from about 50 μm to about 1000 μm, and most preferably from about 100 μm to about 300 μm. While a multiparticulate can have any shape and texture, normally it is spherical with a smooth surface. These physical characteristics lead to excellent flow properties, improved “mouth feel,” ease of swallowing and ease of uniform coating. Such multiparticulates of azithromycin are particularly suitable for administration of single doses of the drug inasmuch as a relatively large amount of the drug can be delivered at a controlled rate over a relatively long period of time.

### Azithromycin Cores

“Azithromycin” means all amorphous and crystalline forms of azithromycin including all polymorphs, isomorphs, clathrates, salts, solvates and hydrates of azithromycin, as well as anhydrous azithromycin, or a combination of forms. Preferably, the azithromycin of the present invention is azithromycin dihydrate which is disclosed in U.S. Pat. No. 6,268,489 B1. In alternate embodiments of the present invention, the azithromycin comprises a non-dihydrate azithromycin, a mixture of non-dihydrate azithromycins, or a mixture of azithromycin dihydrate and non-dihydrate azithromycins.

The term “core” as used herein is defined as the central portion of the composition, such as a particle, granule, or bead, that is subsequently coated with a coating material. A core of the present invention comprises azithromycin. Preferably, the core further comprises a carrier. The term “carrier” refers to pharmaceutically acceptable materials primarily used as a matrix for the core or to control for the rate of azithromycin release from the core, or as both. The carrier may be a single material or a mixture of two or more materials. When the core comprises azithromycin and a carrier, preferably, the azithromycin makes up about 10 wt % to about 95 wt % of the total weight of the core. More preferably, the azithromycin makes up about 20 wt % to about 90 wt % of the core, and even more preferably, at least about 40 wt % to about 70 wt % of the core.

To minimize the potential for changes in the physical characteristics of the multiparticulates over time, especially when stored at elevated temperatures, it is preferred that the carrier be solid at a temperature of at least about 40° C. More preferably, the carrier should be solid at a temperature of at least about 50° C. and even more preferably of at least about 60° C.

Examples of carriers suitable for use in the cores of the present invention include waxes, such as synthetic wax, microcrystalline wax, paraffin wax, Carnauba wax, and beeswax; glycerides, such as glyceryl monooleate, glyceryl monostearate, glyceryl palmitostearate, polyethoxylated castor oil derivatives, hydrogenated vegetable oils, a glyceryl behenate, glyceryl tristearate, glyceryl tripalmitate; long-chain alcohols, such as stearyl alcohol, cetyl alcohol, and polyethylene glycol; and mixtures thereof. Preferably, the carrier comprises a glyceride having at least one alkylate substituent of 16 or more carbon atoms. More preferably, the carrier comprises a glyceryl behenate.

In a more preferred embodiment, the azithromycin cores comprise azithromycin, a carrier and a dissolution enhancer. The carrier and the dissolution enhancer function as a matrix for the core or to control the azithromycin release rate from the core, or both. Dissolution enhancer means an excipient, which when included in the cores, results in a faster rate of release of azithromycin than that provided by a control core containing the same amount of azithromycin without the dissolution enhancer. Generally, the rate of release of azithromycin from the cores increases with increasing amounts of dissolution enhancers. Such agents generally have a high water solubility and are often surfactants or wetting agents that can promote solubilization of other excipients in the composition. Typically, the weight percentage of dissolution enhancer present in the core is less than the weight percentage of carrier present in the core.

In one embodiment, the cores of the present invention comprise from about 10 to about 100 wt % azithromycin, from about 0 to about 80 Wt % carrier, and from about 0 wt % to about 30 wt % of a dissolution enhancer, based on the total mass of the core. In another embodiment, the core comprises from about 20 to about 75 wt % azithromycin, from about 25 to about 80 wt % carrier, and from about 0.1 wt % to about 30 wt % of a dissolution enhancer. In yet another embodiment, the core comprises from about 35 to about 55 wt % azithromycin, from about 40 to about 65 wt % of carrier, and from about 1 to about 15 wt % dissolution enhancer.

Examples of suitable dissolution enhancers include, but are not limited to, alcohols such as stearyl alcohol, cetyl alcohol, and polyethylene glycol; surfactants, such as poloxamers (polyoxyethylene polyoxypropylene copolymers, including poloxamer 188, poloxamer 237, poloxamer 338, and poloxamer 407), docusate salts, polyoxyethylene alkyl ethers, polyoxyethylene castor oil derivatives, polyoxyethylene sorbitan fatty acid esters, sorbitan esters, alkyl sulfates (such as sodium lauryl sulfate), polysorbates, and polyoxyethylene alkyl esters; ether-substituted cellulosics, such as hydroxypropyl cellulose and hydroxypropyl methyl cellulose; sugars such as glucose, sucrose, xylitol, sorbitol, and maltitol; salts such as sodium chloride, potassium chloride, lithium chloride, calcium chloride, magnesium chloride, sodium sulfate, potassium sulfate, sodium carbonate, magnesium sulfate, and potassium phosphate; amino acids such as alanine and glycine; and mixtures thereof. Preferably, the dissolution enhancer comprises a surfactant.

More preferably, the dissolution enhancer comprises a poloxamer. Poloxamers are a series of closely related block copolymers of ethylene oxide and propylene oxide. Preferably, the poloxamer is Poloxamer 407 which is described in the exemplification herein.

In this embodiment wherein the core further comprises a dissolution enhancer, it is further preferred that the carrier is selected from the group consisting of waxes, such as synthetic wax, microcrystalline wax, paraffin wax, Carnauba wax, and beeswax; glycerides, such as glyceryl monooleate, glyceryl monostearate, glyceryl palmitostearate, polyethoxylated castor oil derivatives, hydrogenated vegetable oils, glyceryl mono-, di- or tribehenates, glyceryl tristearate, glyceryl tripalmitate; and mixtures thereof.

Azithromycin can potentially react with carriers, and optional excipients, such as dissolution enhancers, which have acidic or ester groups to form esters of azithromycin. Carriers and excipients may be characterized as having “low reactivity,” “medium reactivity,” and “high reactivity” to form azithromycin esters.

Examples of low reactivity carriers and optional excipients include long-chain alcohols, such as stearyl alcohol, cetyl alcohol, and polyethylene glycol; poloxamers; ethers, such as polyoxyethylene alkyl ethers; ether-substituted cellulosics, such as microcrystalline cellulose, hydroxypropyl cellulose, hydroxypropyl methyl cellulose, and ethylcellulose; sugars such as glucose, sucrose, xylitol, sorbitol, and maltitol; and salts such as sodium chloride, potassium chloride, lithium chloride, calcium chloride, magnesium chloride, sodium sulfate, potassium sulfate, sodium carbonate, magnesium sulfate, and potassium phosphate.

Moderate reactivity carriers and optional excipients often contain acid or ester substituents, but relatively few as compared to the molecular weight of the carrier or optional excipient. Examples include long-chain fatty acid esters, such as glyceryl monooleate, glyceryl monostearate, glyceryl palmitostearate, polyethoxylated castor oil derivatives, hydrogenated vegetable oils, glyceryl dibehenate, and mixtures of mono-, di-, and tri-alkyl glycerides; glycolized fatty acid esters, such as polyethylene glycol stearate and polyethylene glycol distearate; polysorbates; and waxes, such as Carnauba wax and white and yellow beeswax. Glyceryl behenate, as defined herein, comprises glyceryl monobehenate, glyceryl dibehenate, glyceryl tribehenate, or a mixture of any two or all three of said glyceryl mono-, di- and tribehenates.

Highly reactive carriers and optional excipients usually have several acid or ester substituents or low molecular weights. Examples include carboxylic acids such as stearic acid, benzoic acid, citric acid, fumaric acid, lactic acid, and maleic acid; short to medium chain fatty-acid esters, such as isopropyl palmitate, isopropyl myristate, triethyl citrate, lecithin, triacetin, and dibutyl sebacate; ester-substituted cellulosics, such as cellulose acetate, cellulose acetate phthalate, hydroxypropyl methyl cellulose phthalate, cellulose acetate trimellitate, and hydroxypropyl methyl cellulose acetate succinate; and acid or ester functionalized polymethacrylates and polyacrylates. Generally, the acid/ester concentration on highly reactive carriers and optional excipients is so high that if these carriers and optional excipients come into direct contact with azithromycin in the formulation, unacceptably high concentrations of azithromycin esters form during processing or storage of the composition. Thus, such highly reactive carriers and optional excipients are preferably only used in combination with a carrier or optional excipient with lower reactivity so that the total amount of acid and ester groups on the carrier and optional excipients used in the multiparticulate is low.

The azithromycin cores of the present invention should have a low concentration of azithromycin esters, meaning the concentration of azithromycin esters in the core relative to the total weight of azithromycin originally present in the core should be less than 5 wt %, preferably less than 1 wt %; and more preferably less than 0.5 wt %.

To obtain cores with an acceptable amount of azithromycin esters (i.e. less than about 5 wt %), there is a trade-off relationship between the concentration of acid and ester substituents on the carrier and the crystallinity of azithromycin in the core. The greater the crystallinity of azithromycin in the core, the greater the degree of the carrier's acid/ester substitution may be to obtain a core with acceptable amounts of azithromycin esters. This relationship may be quantified by the following mathematical expression:

[A]≦0.2/(1−x) (I)

where [A] is the total concentration of acid/ester substitution on the carrier and optional excipients in meq/g azithromycin and is less than or equal to 2 meq/g, and x is the weight fraction of the azithromycin in the composition that is crystalline. When the carrier and optional excipients comprises more than one excipient, the value of [A] refers to the total concentration of acid/ester substitution on all the excipients that make up the carrier and optional excipients, in units of meq/g azithromycin.

For more preferable cores having less than about 1 wt % azithromycin esters, the azithromycin, carrier, and optional excipients will satisfy the following expression:

[A]≦0.04/(1−x). (II)

For more preferable cores having less than about 0.5 wt % azithromycin esters, the azithromycin, carrier, and optional excipients will satisfy the following expression:

[A]≦0.02/(1−x). (III)

The crystallinity of azithromycin in the core and the trade-off between the carrier's and optional excipient's degree of acid/ester substitution can be determined from the foregoing mathematical expressions (I)-(II).

From the standpoint of reactivity to form azithromycin esters, the dissolution enhancers preferably have a concentration of acid/ester substituents of less than about 0.13 meq/g azithromycin present in the composition. Preferably, the dissolution enhancer has a concentration of acid/ester substituents of less than about 0.10 meq/g azithromycin, more preferably less than about 0.02 meq/g azithromycin, even more preferably less than about 0.01 meq/g, and most preferably less than about 0.002 meq/g.

In addition to having low concentrations of acid and ester substituents, the dissolution enhancer should generally be hydrophilic, such that the rate of release of azithromycin from the bore increases as the concentration of dissolution enhancer in the core increases.

Further description of suitable dissolution enhancers and selection of appropriate excipients for azithromycin multiparticulate cores are disclosed in U.S. Provisional Patent Application Ser. No. 60/527,319 titled “Controlled Release Multiparticulates Formed With Dissolution Enhancers”.

In a yet further preferred embodiment, the cores of the present invention comprise (a) azithromycin; (b) a glyceride carrier having at least one alkylate substituent of 16 or more carbon atoms; and (c) a poloxamer dissolution enhancer. The choice of these particular carrier excipients allows for precise control of the release rate of the azithromycin over a wide range of release rates. Small changes in the relative amounts of the glyceride carrier and the poloxamer result in large changes in the release rate of the drug. This allows the release rate of the drug from the core to be precisely controlled by selecting the proper ratio of drug, glyceride carrier and poloxamer. These materials have the further advantage of releasing nearly all of the drug from the core. Such multiparticulate cores are disclosed more fully in U.S. Provisional Patent Application Ser. No. 60/527,329 titled “Multiparticulate Crystalline Drug Compositions Having Controlled Release Profiles”.

In a further preferred embodiment, the azithromycin dosage form comprises azithromycin cores, comprising about 45 to about 55 wt % azithromycin, about 43 to about 50 wt % glyceryl behenate and about 2 to about 5 wt % poloxamer.

Additional optional excipients may also be included in the azithromycin cores. For example, agents that inhibit or delay the release of azithromycin from the cores can also be included in the carrier. Such dissolution-inhibiting agents are generally hydrophobic. Examples of dissolution-inhibiting agents include hydrocarbon waxes, such as microcrystalline and paraffin wax.

Another useful class of excipients is materials that are used to adjust the viscosity of the molten feed used to form the cores, for example, by a melt-congeal process. Such viscosity-adjusting excipients will generally make up 0 to 25 wt % of the multiparticulate, based on the total mass of the core. The viscosity of the molten feed is a key variable in obtaining cores with a narrow particle size distribution. For example, when a spinning-disc atomizer is employed, it is preferred that the viscosity of the molten mixture be at least about 1 centipoise (cp) and less than about 10,000 cp, more preferably at least 50 cp and less than about 1000 cp. If the molten mixture has a viscosity outside these preferred ranges, a viscosity-adjusting carrier can be added to obtain a molten mixture within the preferred viscosity range. Examples of viscosity-reducing excipients include stearyl alcohol, cetyl alcohol, low molecular weight polyethylene glycol (e.g., less than about 1000 daltons), isopropyl alcohol, and water. Examples of viscosity-increasing excipients include microcrystalline wax, paraffin wax, synthetic wax, high molecular weight polyethylene glycols (e.g., greater than about 5000 daltons), ethyl cellulose, hydroxypropyl cellulose, hydroxypropyl methyl cellulose, methyl cellulose, silicon dioxide, microcrystalline cellulose, magnesium silicate, sugars, and salts.

Other excipients may be added to reduce the static charge on the cores; examples of such anti-static agents include talc and silicon dioxide. Flavorants, colorants, and other excipients may also be added in their usual amounts for their usual purposes.

In one embodiment, the carrier forms a solid solution with one or more optional excipients, meaning that the carrier and one or more optional excipients form a single thermodynamically stable phase. In such cases, excipients that are not solid at a temperature of at least 40° C. can be used, provided the carrier/excipient mixture is solid at a temperature of at least 40° C. This will depend on the melting point of the excipients used and the relative amount of carrier included in the composition.

In another embodiment, the carrier and one or more optional excipients do not form a solid solution, meaning that the carrier and one or more optional excipients form two or more thermodynamically stable phases. In such cases, the carrier/excipient mixture may be entirely molten at the processing temperatures used to form cores or one material may be solid while the other(s) are molten, resulting in a suspension of one material in the molten mixture.

When the carrier and one or more optional excipients do not form a solid solution but a solid solution is desired, for example, to obtain a specific release profile, an additional excipient may be included in the composition to produce a solid solution comprising the carrier, the one or more optional excipients, and the additional excipient. For example, it may be desirable to use a carrier comprising microcrystalline wax and a poloxamer to obtain a multiparticulate with the desired release profile. In such cases a solid solution is not formed, in part due to the hydrophobic nature of the microcrystalline wax and the hydrophilic nature of the poloxamer. By including a small amount of a third excipient, such as stearyl alcohol, in the formulation, a solid solution can be obtained, resulting in a core with the desired release profile.

In an alternate embodiment, the cores are in the form of a non-disintegrating matrix. By “non-disintegrating matrix” is meant that at least a portion of the carrier does not dissolve or disintegrate after introduction of the cores to an aqueous use environment. In such cases, the azithromycin and optionally a portion of one or more of the carriers, for example, a dissolution enhancer, are removed from the core by dissolution. At least a portion of the carrier does not dissolve or disintegrate and is excreted when the use environment is in vivo, or remains suspended in a test solution when the use environment is in vitro. In this aspect, it is preferred that at least a portion of the carrier have a low solubility in the aqueous use environment. Preferably, the solubility of at least a portion of the carrier in the aqueous use environment is less than about 1 mg/mL, more preferably less than about 0.1 mg/mL, and most preferably less than about 0.01 mg/ml. Examples of suitable low-solubility carriers include waxes, such as synthetic wax, microcrystalline wax, paraffin wax, Carnauba wax, and beeswax; glycerides, such as glyceryl monooleate, glyceryl monostearate, glyceryl palmitostearate, glyceryl behenates, glyceryl tristearate, glyceryl tripalmitate; and mixtures thereof.

Preferably, the core is made such that the amount of azithromycin present on the exterior of the core is minimized. In one embodiment, less than 10 wt % of the azithromycin present in the core is present on the exterior surface of the core. Such cores may be made using the thermal or liquid-based processed described herein below. In a preferred embodiment, such cores are made using a melt-congeal process as described herein.

The azithromycin cores of the present invention generally have a mean diameter of less than about 5000 μm. In a preferred embodiment, the mean diameter of the cores ranges from about 50 to about 3000 μm and more preferably from about 100 to about 300 μm. Note that the diameter of the cores can be used to adjust the release rate of azithromycin from the cores. Generally, the smaller the diameter of the cores, the faster will be the azithromycin release rate from a particular formulation. This is because the overall surface area in contact with the dissolution medium increases as the diameter of the core decreases. Thus, adjustments in the mean diameter of the cores can be used to adjust the azithromycin release profile.

In one embodiment, the core comprises a mixture of azithromycin with one or more excipients selected to form a matrix capable of limiting the dissolution rate of the azithromycin into an aqueous medium. The matrix materials useful for this embodiment are generally water-insoluble materials such as waxes, cellulose, or other water-insoluble polymers. If needed, the matrix materials may optionally be formulated with water-soluble materials which can be used as binders or as permeability-modifying agents. Matrix materials useful for the manufacture of these dosage forms include microcrystalline cellulose such as Avicel (registered trademark of FMC Corp., Philadelphia, Pa.), including grades of microcrystalline cellulose to which binders such as hydroxypropyl methyl cellulose have been added, waxes such as paraffin, modified vegetable oils, carnauba wax, hydrogenated castor oil, beeswax, and the like, as well as synthetic polymers such as poly(vinyl chloride), poly(vinyl acetate), copolymers of vinyl acetate and ethylene, polystyrene, and the like. Water soluble binders or release modifying agents which can optionally be formulated into the matrix include water-soluble polymers such as hydroxypropyl cellulose (HPC), hydroxypropyl methyl cellulose (HPMC), methyl cellulose, poly(N-vinyl-2-pyrrolidinone) (PVP), poly(ethylene oxide) (PEO), poly(vinyl alcohol) (PVA), xanthan gum, carrageenan, and other such natural and synthetic materials. In addition, materials which function as release-modifying agents include water-soluble materials such as sugars or salts. Preferred water-soluble materials include lactose, sucrose, glucose, and mannitol, as well as HPC, HPMC, and PVP.

The azithromycin cores of the present invention can be made by any known process that results in particles, containing azithromycin and a carrier, with the desired size and release rate characteristics for the azithromycin. Preferred processes for forming such cores include thermal-based processes, such as melt- and spray-congealing; liquid-based processes, such as extrusion/spheronization, wet granulation, spray-coating, and spray-drying; and other granulation processes such as dry granulation and melt granulation.

Another process for manufacturing the azithromycin cores is the preparation of wax granules. In this process, a desired amount of azithromycin is stirred with liquid wax to form a homogeneous mixture, cooled and then forced through a screen to form granules. Preferred matrix materials are waxy substances. Especially preferred are hydrogenated castor oil and carnauba wax and stearyl alcohol.

The azithromycin cores may be made by a melt-congeal process comprising the steps of (a) forming a molten mixture comprising azithromycin and a pharmaceutically acceptable carrier; (b) delivering the molten mixture of step (a) to an atomizing means to form droplets from the molten mixture; and (c) congealing the droplets from step (b) to form the cores.

When using thermal-based processes, such as the melt-congeal process, to make the azithromycin cores of the present invention, the heat transfer to the azithromycin is minimized to prevent significant thermal degradation of the azithromycin during the process. It is also preferred that the carrier have a melting point that is less then the melting point of azithromycin. For example, azithromycin dihydrate has a melting point of 113° C. to 150°. Thus, when azithromycin dihydrate is used in the cores of the present invention, it is preferred that the carrier have a melting point that is less than about 113° C. As used herein, the term “melting point of the carrier” or “Tₘ” means the temperature at which the carrier, when containing the drug and any optional excipients present in the multiparticulate, transitions from its crystalline to its liquid state. When the carrier is not crystalline, “melting point of the carrier” means the temperature at which the carrier becomes fluid in the sense that it will flow when subjected to one or more forces such as pressure, shear, and centrifugal force, in a manner similar to a crystalline material in the liquid state.

The azithromycin in the molten mixture may be dissolved in the molten mixture, may be a suspension of crystalline azithromycin distributed in the molten mixture, or any combination of such states or those states that are in between. Preferably, the molten mixture comprises a homogeneous suspension of crystalline azithromycin in the molten carrier where the fraction of azithromycin that melts or dissolves in the molten carrier is kept relatively low. Preferably less than about 30 wt % of the total azithromycin melts or dissolves in the molten carrier. It is preferred that the azithromycin be present as the crystalline dihydrate.

Thus, by “molten mixture” is meant that the mixture of azithromycin and carrier are heated sufficiently that the mixture becomes sufficiently fluid that the mixture may be formed into droplets or atomized. Atomization of the molten mixture may be carried out using any of the atomization methods described below. Generally, the mixture is molten in the sense that it will flow when subjected to one or more forces such as pressure, shear, and centrifugal force, such as that exerted by a centrifugal or spinning-disk atomizer. Thus, the azithromycin/carrier mixture may be considered “molten” when any portion of the carrier and azithromycin become fluid such that the mixture, as a whole, is sufficiently fluid that it may be atomized. Generally, a mixture is sufficiently fluid for atomization when the viscosity of the molten mixture is less than about 20,000 cp, preferably less than about 15,000 cp, more preferably less than about 10,000 cp. Often, the mixture becomes molten when the mixture is heated above the melting point of one or more of the carrier components, in cases where the carrier is sufficiently crystalline to have a relatively sharp melting point; or, when the carrier components are amorphous, above the softening point of one or more of the carrier components. Thus, the molten mixture is often a suspension of solid particles in a fluid matrix. In one preferred embodiment, the molten mixture comprises a mixture of substantially crystalline azithromycin particles suspended in a carrier that is substantially fluid. In such cases, a portion of the azithromycin may be dissolved in the fluid carrier and a portion of the carrier may remain solid.

Although the term “melt” refers specifically to the transition of a crystalline material from its crystalline to its liquid state, which occurs at its melting point, and the term “molten” refers to such a crystalline material in its liquid state, as used herein, the terms are used more broadly, referring in the case of “melt” to the heating of any material or mixture of materials sufficiently that it becomes fluid in the sense that it may be pumped or atomized in a manner similar to a crystalline material in the liquid state. Likewise “molten” refers to any material or mixture of materials that is in such a fluid state.

Virtually any process can be used to form the molten mixture. One method involves melting the carrier in a tank, adding the azithromycin to the molten carrier, and then mixing the mixture to ensure the azithromycin is uniformly distributed therein. Alternatively, both the azithromycin and carrier may be added to the tank and the mixture heated and mixed to form the molten mixture. When the carrier comprises more than one material, the molten mixture may be prepared using two tanks, melting a first carrier in one tank and a second in another. The azithromycin is added to one of these tanks and mixed as described above. In another method, a continuously stirred tank system may be used, wherein the azithromycin and carrier are continuously added to a heated tank equipped with means for continuous mixing, while the molten mixture is continuously removed from the tank.

The molten mixture may also be formed using a continuous mill, such as a Dyno® Mill (W. A. Bachofen of Switzerland). The azithromycin and carrier are typically fed to the continuous mill in solid form, entering a grinding chamber containing grinding media, such as beads 0.25 to 5 mm in diameter. The grinding chamber typically is jacketed so heating or cooling fluid may be circulated around the chamber to control its temperature. The molten mixture is formed in the grinding chamber, and exits the chamber through a separator to remove the grinding media.

An especially preferred method of forming the molten mixture is by an extruder. By “extruder” is meant a device or collection of devices that creates a molten extrudate by heat and/or shear forces and/or produces a uniformly mixed extrudate from a solid and/or liquid (e.g., molten) feed. Such devices include, but are not limited to single-screw extruders; twin-screw extruders, including co-rotating, counter-rotating, intermeshing, and non-intermeshing extruders; multiple screw extruders; ram extruders, consisting of a heated cylinder and a piston for extruding the molten feed; gear-pump extruders, consisting of a heated gear pump, generally counter-rotating, that simultaneously heats and pumps the molten feed; and conveyer extruders. Conveyer extruders comprise a conveyer means for transporting solid and/or powdered feeds, such, such as a screw conveyer or pneumatic conveyer, and a pump. At least a portion of the conveyer means is heated to a sufficiently high temperature to produce the molten mixture. The molten mixture may optionally be directed to an accumulation tank, before being directed to a pump, which directs the molten mixture to an atomizer. Optionally, an in-line mixer may be used before or after the pump to ensure the molten mixture is substantially homogeneous. In each of these extruders the molten mixture is mixed to form a uniformly mixed extrudate. Such mixing may be accomplished by various mechanical and processing means, including mixing elements, kneading elements, and shear mixing by backflow. Thus, in such devices, the composition is fed to the extruder, which produces a molten mixture that can be directed to the atomizer.

Once the molten mixture has been formed, it is delivered to an atomizer that breaks the molten mixture into small droplets. Virtually any method can be used to deliver the molten mixture to the atomizer, including the use of pumps and various types of pneumatic devices such as pressurized vessels or piston pots. When an extruder is used to form the molten mixture, the extruder itself can be used to deliver the molten mixture to the atomizer. Typically, the molten mixture is maintained at an elevated temperature while delivering the mixture to the atomizer to prevent solidification of the mixture and to keep the molten mixture flowing.

Generally, atomization occurs in one of several ways, including (1) by “pressure” or single-fluid nozzles; (2) by two-fluid nozzles; (3) by centrifugal or spinning-disk atomizers; (4) by ultrasonic nozzles; and (5) by mechanical vibrating nozzles. Detailed descriptions of atomization processes, including how to use spinning disk atomizers to obtain specific particle sizes, can be found in Lefebvre, Atomization and Sprays (1989) or in Perry's Chemical Engineers' Handbook (7th Ed. 1997).

Once the molten mixture has been atomized, the droplets are congealed, typically by contact with a gas or liquid at a temperature below the solidification temperature of the droplets. Typically, it is desirable that the droplets are congealed in less than about 60 seconds, preferably in less than about 10 seconds, more preferably in less than about 1 second. Often, congealing at ambient temperature results in sufficiently rapid solidification of the droplets to avoid excessive azithromycin ester formation. However, the congealing step often occurs in an enclosed space to simplify collection of the cores. In such cases, the temperature of the congealing medium (either gas or liquid) will increase over time as the droplets are introduced into the enclosed space, leading to the possible formation of azithromycin esters. Thus, a cooling gas or liquid is often circulated through the enclosed space to maintain a constant congealing temperature. When the carrier used is highly reactive with azithromycin and the time the azithromycin is exposed to the molten carrier must be limited, the cooling gas or liquid can be cooled to below ambient temperature to promote rapid congealing, thus keeping the formation of azithromycin esters to acceptable levels.

Suitable thermal-based processes are disclosed in detail in U.S. Provisional Patent Application No. 60/527,244 titled “Azithromycin Multiparticulate Dosage Forms by Melt-Congeal Processes”, and U.S. Provisional Patent Application No. 60/527,315 titled “Extrusion Process for Forming Chemically Stable Drug Multiparticulates”.

The azithromycin cores may also be made by a liquid-based process comprising the steps of (a) forming a mixture comprising azithromycin, a pharmaceutically acceptable carrier, and a liquid; (b) forming particles from the mixture of step (a); and (c) removing a substantial portion of the liquid from the particles of step (b) to form the cores. Preferably, step (b) is a method selected from (i) atomization of the mixture, (ii) coating seed cores with the mixture, (iii) wet-granulating the mixture, and (iv) extruding the mixture into a solid mass followed by spheronizing or milling the mass.

Preferably, the liquid has a boiling point of less than about 150° C. Examples of liquids suitable for formation of multiparticulates using liquid-based processes include water; alcohols, such as methanol, ethanol, various isomers of propanol and various isomers of butanol; ketones, such as acetone, methyl ethyl ketone and methyl isobutyl ketone; hydrocarbons, such as pentane, hexane, heptane, cyclohexane, methylcyclohexane, octane and mineral oil; ethers, such as methyl tert-butyl ether, ethyl ether and ethylene glycol monoethyl ether; chlorocarbons, such as chloroform, methylene dichloride and ethylene dichloride; tetrahydrofuran; dimethylsulfoxide; N-methylpyrrolidinone; N,N-dimethylacetamide; acetonitrile; and mixtures thereof.

In one embodiment; the azithromycin cores are formed by atomization of the mixture using an appropriate nozzle to form small droplets of the mixture, which are sprayed into a drying chamber where there is a strong driving force for evaporation of the liquid, to produce solid, generally spherical particles. The strong driving force for evaporation of the liquid is generally provided by maintaining the partial pressure of liquid in the drying chamber well below the vapor pressure of the liquid at the temperature of the particles. This is accomplished by (1) maintaining the pressure in the drying chamber at a partial vacuum (e.g., 0.01 to 0.5 atm); or (2) mixing the droplets with a warm drying gas; or (3) both (1) and (2). Spray-drying processes and spray-drying equipment are described generally in Perry's Chemical Engineers' Handbook, pages 20-54 to 20-57 (6th Ed. 1984).

Alternately, the azithromycin cores are formed by coating the liquid mixture onto seed cores. The seed cores can be made from any suitable material such as starch, microcrystalline cellulose, sugar or wax, by any known method, such as melt- or spray-congealing, extrusion/spheronization, granulation, spray-drying and the like.

The liquid mixture can be sprayed onto such seed cores using coating equipment known in the pharmaceutical arts, such as pan coaters (e.g., Hi-Coater available from Freund Corp. of Tokyo, Japan, Accela-Cota available from Manesty of Liverpool, U.K.), fluidized bed coaters (e.g., Würster coaters or top-spray coaters, available from Glatt Air Technologies, Inc. of Ramsey, N.J. and from Niro Pharma Systems of Bubendorf, Switzerland) and rotary granulators (e.g., CF-Granulator, available from Freund Corp).

In another embodiment, the liquid mixture may be wet-granulated to form the azithromycin cores. Granulation is a process by which relatively small particles are built up into larger granular particles, often with the aid of a carrier, also known as a binder in the pharmaceutical arts. In wet-granulation, a liquid is used to increase the intermolecular forces between particles, leading to an enhancement in granular integrity, referred to as the “strength” of the granule. Often, the strength of the granule is determined by the amount of liquid that is present in the interstitial spaces between the particles during the granulation process. This being the case, it is important that the liquid wet the particles, ideally with a contact angle of zero. Since a large percentage of the particles being granulated are very hydrophilic azithromycin crystals, the liquid needs to be fairly hydrophilic to meet this criterion. Thus, effective wet-granulation liquids tend also to be hydrophilic. Examples of liquids found to be effective wet-granulation liquids include water, ethanol, isopropyl alcohol and acetone. Preferably, the wet-granulation liquid is water at pH 7 or higher.

Several types of wet-granulation processes can be used to form azithromycin-containing cores. Examples include fluidized bed granulation, rotary granulation and high-shear mixers. In fluidized bed granulation, air is used to agitate or “fluidize” particles of azithromycin and/or carrier in a fluidizing chamber. The liquid is then sprayed into this fluidized bed, forming the granules. In rotary granulation, horizontal discs rotate at high speed, forming a rotating “rope” of azithromycin and/or carrier particles at the walls of the granulation vessel. The liquid is sprayed into this rope, forming the granules. High-shear mixers contain an agitator or impeller to mix the particles of azithromycin and/or carrier. The liquid is sprayed into the moving bed of particles, forming granules. In these processes, all or a portion of the carrier can be dissolved into the liquid prior to spraying the liquid onto the particles. Thus, in these processes, the steps of forming the liquid mixture and forming cores from the liquid mixture occur simultaneously.

In another embodiment, the cores are formed by extruding the liquid mixture into a solid mass followed by spheronizing or milling the mass. In this process, the liquid mixture, which is in the form of a paste-like plastic suspension, is extruded through a perforated plate or die to form a solid mass, often in the form of elongated, solid rods. This solid mass is then milled to form the azithromycin cores. In one embodiment, the solid mass is placed, with or without an intervening drying step, onto a rotating disk that has protrusions that break the material into spheres, spheroids, or rounded rods. The so-formed cores are then dried to remove any remaining liquid. This process is sometimes referred to in the pharmaceutical arts as an extrusion/spheronization process.

Once the particles are formed, a portion of the liquid is removed, typically in a drying step, thus forming the multiparticulates. Preferably, at least 80% of the liquid is removed from the particles, more preferably at least 90%, and most preferably at least 95% of the liquid is removed from the particle during the drying step.

Suitable liquid-based processes are disclosed more fully in U.S. Provisional Patent Application Ser. No. 60/527,405 titled “Azithromycin Multiparticulate Dosage Forms by Liquid-Based Processes”.

The azithromycin cores may also be made by a granulation process comprising the steps of (a) forming a solid mixture comprising azithromycin and a pharmaceutically acceptable carrier; and (b) granulating the solid mixture to form the cores. Examples of such granulation processes include dry granulation and melt granulation, both well known in the art. See Remington's Pharmaceutical Sciences (19th Ed. 1995).

An example of a dry granulation process is roller compaction. In roller compaction processes, the solid mixture is compressed between rollers. The rollers can be designed such that the resulting compressed material is in the form of small beads or pellets of the desired diameter. Alternatively, the compressed material is in the form of a ribbon that may be milled to for cores using methods well known in the art. See, for example, Remington's Pharmaceutical Sciences (19th Ed. 1995).

In melt granulation processes, the solid mixture is fed to a granulator that has the capability of heating or melting the carrier. Equipment suitable for use in this process includes high-shear granulators and single or multiple screw extruders, such as those described above for melt-congeal processes. In melt granulation processes, the solid mixture is placed into the granulator and heated until the solid mixture agglomerates. The solid mixture is then kneaded or mixed until the desired particle size is attained. The so-formed granules are then cooled, removed from the granulator and sieved to the desired size fraction, thus forming the azithromycin cores.

In a further embodiment, the core comprises an azithromycin-containing particle coated first with a membrane designed to yield sustained release of the azithromycin. This core is then coated with an enteric coating as described below. The particles contain azithromycin and may contain one or more excipients as needed for fabrication and performance. Particles which contain a high fraction of azithromycin relative to binder are preferred. The particle may be of a composition and be fabricated by any of the techniques previously described.

Sustained release coatings as known in the art may be employed to fabricate the membrane, especially polymer coatings, such as a cellulose ester or ether, an acrylic polymer, or a mixture of polymers. Preferred materials include ethyl cellulose, cellulose acetate and cellulose acetate butyrate. The polymer may be applied as a solution in an organic solvent or as an aqueous dispersion or latex. The coating operation may be conducted in standard equipment such as a fluid bed coater, a Würster boater, or a rotary bed coater, as described herein for enteric coatings. The coating can be non-porous, yet permeable to azithromycin (for example azithromycin may diffuse directly through the membrane), or it may be porous.

If desired, the permeability of the coating may be adjusted by blending of two or more materials. A particularly useful process for tailoring the porosity of the coating comprises adding a pre-determined amount of a finely-divided water-soluble material, such as sugars or salts or water-soluble polymers to a solution or dispersion (e.g., an aqueous latex) of the membrane-forming polymer to be used. When the dosage form is ingested into the aqueous medium of the GI tract, these water soluble membrane additives are leached out of the membrane, leaving pores which facilitate release of the drug. The membrane coating can also be modified by the addition of plasticizers, as known in the art.

A particularly useful variation of the process for applying a membrane coating comprises dissolving the coating polymer in a mixture of solvents chosen such that as the coating dries, a phase inversion takes place in the applied coating solution, resulting in a membrane with a porous structure. Numerous examples of this type of coating system are given in European Patent Specification 0 357 369 B1, published Mar. 7, 1990.

In order to reduce the formation of azithromycin esters, preferably, at least 70 wt % of the azithromycin in the core is crystalline. More preferably, at least 80 wt % of the azithromycin is crystalline. Even more preferably, at least 90 wt % of the azithromycin is crystalline. Most preferably, at least 95 wt % of the azithromycin is crystalline. Crystalline azithromycin is preferred since it is more chemically and physically stable than the amorphous form or dissolved azithromycin.

The crystallinity of the azithromycin may be determined using Powder X Ray Diffraction (PXRD) analysis. In an exemplary procedure, PXRD analysis may be performed on a Bruker AXS D8 Advance diffractometer. In this analysis, samples of about 500 mg are packed in Lucite sample cups and the sample surface smoothed using a glass microscope slide to provide a consistently smooth sample surface that is level with the top of the sample cup. Samples are spun in the φ plane at a rate of 30 rpm to minimize crystal orientation effects. The X-ray source (S/B KCu_{α}, λ=1.54 Å) is operated at a voltage of 45 kV and a current of 40 mA. Data for each sample are collected over a period of about 20 to about 60 minutes in continuous detector scan mode at a scan speed of about 1.8 seconds/step to about 12 seconds/step and a step size of 0.02/step. Diffractograms are collected over the 2θ range of about 10 to 16.

The crystallinity of the test sample is determined by comparison with calibration standards as follows. The calibration standards consist of physical mixtures of 20 wt %/80 wt % azithromycin/carrier, and 80 wt %/20 wt % azithromycin/carrier. Each physical mixture is blended together 15 minutes on a Turbula mixer. Using the instrument software, the area under the diffractogram curve is integrated over the 2θ range of 10° to 16° using a linear baseline. This integration range includes as many azithromycin-specific peaks as possible while excluding carrier-related peaks. In addition, the large azithromycin-specitic peak at approximately 10 2θ is omitted due to the large scan-to-scan variability in its integrated area. A linear calibration curve of percent crystalline azithromycin versus the area under the diffractogram curve is generated from the calibration standards. The crystallinity of the test sample is then determined using these calibration results and the area under the curve for the test sample. Results are reported as a mean percent azithromycin crystallinity (by crystal mass).

While the azithromycin in the cores can be amorphous or crystalline, it is preferred that a substantial portion of the azithromycin is crystalline, preferably the crystalline dihydrate. By “substantial portion” is meant that at least 80% of the azithromycin is crystalline. The crystalline form is preferred because it tends to result in cores with improved chemical and physical stability.

One key to maintaining the crystalline form of azithromycin during formation of cores via thermal-based and liquid-based processes is to maintain a high activity of water and any solvate solvents in the carrier, atmosphere or gas with which the composition comes in contact. The activity of water or solvent should be equivalent to or greater than that in the crystalline state. This will ensure that the water or solvent present in the crystal form of azithromycin remains at equilibrium with the atmosphere, thus preventing a loss of hydrated water or solvated solvent. For example, if the process for forming the cores requires that crystalline azithromycin, the crystalline dihydrate, for instance, be exposed to high temperatures (e.g., during a melt- or spray-congeal process), the atmosphere near the azithromycin should be maintained at high humidity to limit the loss of the hydrated water from the azithromycin crystals, and thus a change in the crystalline form of the azithromycin.

The humidity level required is that equivalent to or greater than the activity of water in the crystalline state. This can be determined experimentally, for example, using a dynamic vapor sorption apparatus. In this test, a sample of the crystalline azithromycin is placed in a chamber and equilibrated at a constant temperature and relative humidity. The weight of the sample is then recorded. The weight of the sample is then monitored as the relative humidity of the atmosphere in the chamber is decreased. When the relative humidity in the chamber decreases to below the level equivalent to the activity of water in the crystalline state, the sample will begin to loose weight as waters of hydration are lost. Thus, to maintain the crystalline state of the azithromycin, the humidity level should be maintained at or above the relative humidity at which the azithromycin begins to lose weight. A similar test can be used to determine the appropriate amount of solvent vapor required to maintain a crystalline solvate form of azithromycin.

When crystalline azithromycin, such as the dihydrate form, is added to a molten carrier, a small amount of water, on the order of 30 to 100 wt % of the solubility of water in the molten carrier at the process temperature can be added to the carrier to ensure there is sufficient water to prevent loss of the azithromycin dihydrate crystalline form.

Likewise, if a liquid-based process is used to form the composition, the liquid should contain sufficient water (e.g., 30 to 100 wt % the solubility of water in the liquid) to prevent a loss of the waters from hydrated crystalline azithromycin. In addition, the atmosphere near the azithromycin during any drying steps to remove the liquid should be humidified sufficiently to prevent the loss of water and thereby maintain the crystalline dihydrate form. Generally, the higher the processing temperature, the higher the required concentration of water vapor or solvent in the carrier, atmosphere, or gas to which the azithromycin is exposed to maintain the hydrated or solvated form of the azithromycin.

Processes to maintain the crystalline form of azithromycin while forming azithromycin cores or uncoated azithromycin multiparticulates are disclosed more fully in U.S. Provisional Patent Application Ser. No. 60/527,316 titled “Method for Making Pharmaceutical Multiparticulates”.

The azithromycin cores of the present invention may be post-treated to improve the drug crystallinity and/or the stability of the multiparticulate. In one embodiment, the cores comprise azithromycin and a carrier, wherein the carrier, when in the core, has a melting point of Tₘ in ° C.; the cores are treated after formation by at least one of (i) heating the cores to a temperature of at least 35° C. but less than (Tₘ° C.-10° C.), and (ii) exposing the cores to a mobility-enhancing agent. Such a post-treatment step results in an increase in drug crystallinity in the cores, and typically an improvement in at least one of the chemical stability, physical stability, and dissolution stability of the cores. Post-treatment processes are disclosed more fully in U.S. Provisional Patent Application Ser. No. 60/527,245, titled “Multiparticulate Compositions with Improved Stability”.

Preferably, wherein the azithromycin cores comprise about 45 to about 55 wt % azithromycin, about 43 to about 50 wt % glyceryl behenate and about 2 to about 5 wt % poloxamer, the azithromycin cores are post-treated by maintaining them at a temperature of about 40° C. at a relative humidity of about 75%, or sealed with water in a container maintained at 40° C., for 2 days or more.

More preferably, wherein the azithromycin cores comprise about 50 wt % azithromycin dihydrate, about 46 to about 48 wt % Compritol® 888 ATO, and about 2 to about 4 wt % Lutrol® F127 NF, the azithromycin cores are post-treated by maintaining them at a temperature of about 40° C. at a relative humidity of about 75%, or sealed with water in a container maintained at 40° C., for about 2 days or more.

### Formation of Azithromycin Esters

The inventors have discovered that azithromycin degradants can be in the form of azithromycin esters. The inventors further discovered that azithromycin esters can form by interaction of azithromycin with the coating material or with excipients used in the coating formulation. Azithromycin esters have been discovered to form either through direct esterification or transesterification of the hydroxyl substituents of azithromycin. By direct esterification is meant that a coating having an acid substituent can react with the hydroxyl substituents of azithromycin to form an azithromycin ester. By “acid substituent” is meant any of a carboxylic acid, sulfonic acid, or phosphoric acid substituent. By transesterification is meant that a coating having an ester substituent, i.e., carboxylic acid esters, sulfonyl esters, or phosphotyl esters, can react with the hydroxyl group of the azithromycin, transferring the carboxylate of the coating to azithromycin, thus resulting in formation of an azithromycin ester. Typically, in such reactions, one acid or one ester substituent on the coating can each react with one molecule of azithromycin, although formation of two or more esters on a single molecule of azithromycin is possible.

Since the azithromycin dosage forms may be stored for up to two years or even longer prior to dosing, it is preferred that the amount of azithromycin esters in the stored dosage form not exceed the above values prior to dosing.

Processes for reducing ester formation during core formation are described in more detail in commonly assigned U.S. Provisional Patent Application Ser. Nos. 60/527,244 titled “Azithromycin Multiparticulate Dosage Forms by Melt-Congeal Processes”, 60/527,319 titled “Controlled Release Multiparticulates Formed with Dissolution Enhancers”, and 60/527,405 titled “Azithromycin Multiparticulate Dosage Forms by Liquid-Based Processes”.

### Rates of Ester Formation

The inventors have found that the rate of azithromycin ester formation Rₑ in wt %/day may be predicted using a zero-order reaction model, according to the following Equation IV:

Rₑ=Cₑₛₜₑᵣₛ÷t (IV)

where Cₑₛₜₑᵣₛ is the concentration of azithromycin esters formed (wt %), and t is time of contact between azithromycin and the coating in days at temperature T (° C.). This equation is suitable for determining Rₑ when Cₑₛₜₑᵣₛ is less than about 30 wt %.

A variety of azithromycin esters may be formed by reaction of the coating excipients with azithromycin. Unless otherwise stated, Cₑₛₜₑᵣₛ generally refers to the concentration of all azithromycin esters combined.

To determine the reaction rate for forming azithromycin esters with the coating, a blend of the coating materials with azithromycin is formed and then stored at a temperature from about 20° C. to about 50° C. Samples of the blend are periodically removed and analyzed for azithromycin esters, as described below. The reaction rate for formation of azithromycin esters is then determined using Equation IV.

One method of analyzing a composition for azithromycin esters is by high performance liquid chromatography mass spectrometer (LCMS) analysis which combines a high-performance liquid chromatograph (HPLC), to separate the various species, and a mass spectrometer (MS) to detect the species. In this method, the azithromycin and any azithromycin esters are extracted from the multiparticulates using an appropriate solvent, such as methanol or isopropyl alcohol. The extraction solvent may then be filtered with a 0.45 μm nylon syringe filter to remove any particles present in the solvent. The various species present in the extraction solvent can then be separated by HPLC using procedures well known in the art. A mass spectrometer is used to detect species, with the concentrations of azithromycin and azithromycin esters being calculated from the mass spectrometer peak areas based on either an internal or external azithromycin control. Preferably, if authentic standards of the esters have been synthesized, external references to the azithromycin esters may be used. The azithromycin ester value is then reported as a percentage of the total azithromycin in the sample.

Compositions of the present invention have less than about 5 wt % total azithromycin esters after storage for 2 years at ambient temperature and humidity or, under ICH guidelines, 25° C. and 60 relative humidity (RH) relative to the total weight of azithromycin originally present in the composition. Preferred embodiments of the invention have less than about 1 wt % azithromycin esters after such storage, more preferably less than about 0.5 wt %, and most preferably less than about 0.1 wt %.

Accelerated storage tests can be performed following International Conference on Harmonization (ICH) guidelines. Under these guidelines, a simulation of two years at ambient temperature is conducted by measuring the ester formation of a sample stored for one year at 30° C./60% relative humidity (RH). More rapid simulations can be conducted by storing the sample for six months at 40° C./75% RH.

### Enteric Coatings

The pharmaceutical compositions of the present invention comprises a pharmaceutically acceptable enteric coating disposed upon the azithromycin core. The term “disposed upon” as used herein means that the coating substantially surrounds, covers, or encapsulates the azithromycin core. An enteric coating is a pH-sensitive coating which is substantially insoluble and impermeable at a pH of the stomach and which is more soluble and permeable at the pH of the small intestine. Preferably, the enteric coating is substantially insoluble and impermeable at pH<5.0, and becomes water-soluble at a pH above 5.0. All materials used in the application of the enteric coating to the cores, including any coating polymers, plasticizers, additives, and solvents, are simply referred to as the “coating”.

Enteric polymers which are relatively insoluble and impermeable at the pH of the stomach, but which are more soluble and permeable at the pH of the small intestine and colon include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, cellulose acetate trimellitate, hydroxypropyl methylcellulose acetate succinate, cellulose acetate succinate, carboxymethyl cellulose, carboxyethyl cellulose, carboxymethyl ethyl cellulose, styrene and maleic acid copolymers, polyacrylic acid derivatives such as acrylic acid and acrylic ester copolymers, polymethacrylic acid and esters thereof, poly acrylic methacrylic acid copolymers, shellac, vinyl acetate and crotonic acid copolymers, and mixtures thereof.

Cellulose acetate phthalate (CAP) may be applied to azithromycin cores to provide delayed release of azithromycin until the azithromycin-containing multiparticulate has passed the sensitive duodenal region, that is to delay the release of azithromycin in the gastrointestinal tract until about 15 minutes, and preferably about 30 minutes, after the azithromycin-containing multiparticulate has passed from the stomach to the duodenum. The CAP coating solution may also contain one or more plasticizers, such as diethyl phthalate, polyethyleneglycol-400, triacetin, triacetin citrate, propylene glycol, and others as known in the art. Preferred plasticizers are diethyl phthalate and triacetin. The CAP coating formulation may also contain one or more emulsifiers, such as polysorbate-80.

Anionic acrylic copolymers of methacrylic acid and methylmethacrylate are also particularly useful coating materials for delaying the release of azithromycin from azithromycin-containing cores until the multiparticulates have moved to a position in the small intestine which is distal to the duodenum. Copolymers of this type are available from RohmPharma Corp, under the tradenames Eudragit-L® and Eudragit-S®. Eudragit-L® and Eudragit-S® are anionic copolymers of methacrylic acid and methylmethacrylate. The ratio of free carboxyl groups to the esters is approximately 1:1 in Eudragit-L® and approximately 1:2 in Eudragit-S®. Mixtures of Eudragit-L® and Eudragit-S® may also be used. For coating of azithromycin-containing cores, these acrylic coating polymers may be dissolved in an organic solvent or mixture of organic solvents, or formed into an aqueous dispersion, known in the art as a latex formulation. Useful solvents for this purpose are acetone, isopropyl alcohol, water, methylene chloride, and mixtures thereof. It is generally advisable to include 5-20% plasticizer in coating formulations of acrylic copolymers. Useful plasticizers are polyethylene glycols, propylene glycols, diethyl phthalate, dibutyl phthalate, castor oil, triethyl citrate, and triacetin.

The delay time before release of azithromycin, after the coated multiparticulate has exited the stomach, may be controlled by choice of the relative amounts of Eudragit-L® and Eudragit-S® in the coating, and by choice of the coating thickness. Eudragit-L® films dissolve above pH 6.0, and Eudragit-S® films dissolve above 7.0, and mixtures dissolve at intermediate pH's. Since the pH of the duodenum is approximately 6.0 and the pH of the colon is approximately 7.0, coatings composed of mixtures of Eudragit-L® and Eudragit-S® provide protection of the duodenum from azithromycin. In order to delay the release of azithromycin for about 15 minutes or more, preferably 30 minutes or more, after the multiparticulate has exited the stomach, preferred coatings comprise from about 9:1 to about 1:9 Eudragit-L®/Eudragit-S®, more preferably from about 9:1 to about 1:4 Eudragit-L®/Eudragit-S®. The coating may comprise from about 3% to about 200% of the weight of the uncoated core. Preferably, the coating comprises from about 5% to about 100% of the weight of the uncoated core. In one embodiment, the enteric coating material comprises (i) a copolymer of methacrylic acid and ethyl acrylate and (ii) triethyl citrate. Preferably, the enteric coating material should not cause significant production of azithromycin esters. To satisfy a total azithromycin esters content of less than about 5 wt %, the coating material, including excipients and additives, is selected such that the composition will have a rate of azithromycin ester formation Rₑ in wt %/day of Rₑ≦1.8×10⁸·e^{-7070/(T+273)} where T is in ° C.

To satisfy a preferred total azithromycin esters content of less than about 1 wt %, the coating is selected such that composition will have a rate of ester formation of Rₑ≦3.6×10⁷·e^{-7070/(T+273)} where T is in ° C.

To satisfy the more preferred total azithromycin esters content of less than about 0.5 wt %, the coating is selected such that composition will have a rate of ester formation of Rₑ≦1.8×10⁷·e^{-7070/(T+273)} where T is in ° C.

The reactivity of an enteric coating material will depend on the nature of the reactive substituents and on the molecular weight of the material. When the material has a high molecular weight (i.e., >2000 daltons), the material will generally have a low reactivity with azithromycin. Preferably, the coating material's molecular weight is >5000 daltons, and more preferably >10,000 daltons. Examples of coating materials include carboxymethyl cellulose, carboxyethyl cellulose, carboxymethyl ethyl cellulose, styrene and maleic acid copolymers, polyacrylic acid derivatives such as acrylic acid and acrylic ester copolymers, polymethacrylic acid, polyacrylic and methacrylic acid copolymers, crotonic acid copolymers, and mixtures thereof.

In addition, enteric coating materials with stable ester linkages also have fairly low reactivity with azithromycin such as hydroxypropylmethyl cellulose acetate succinate.

Impurities present in the coating materials, additives used in producing the coating or degradation products from the coating may also be reactive with azithromycin. Additives such as plasticizers can be extremely reactive with azithromycin. Thus, any coating candidate should be screened to ensure it does not contain an undesirable amount of a species that can potentially react with azithromycin to form azithromycin esters.

Other enteric coating materials, such as those that contain phthalate substituents, trimellitate substituents or a molecular weight of <2000 daltons, are highly reactive with azithromycin and could result in the formation of undesirable azithromycin esters. To use reactive enteric coatings in the present invention, it is preferable that the azithromycin in the core be isolated from the reactive coating materials such that they generally do not dome into physical contact. This can be achieved by, for example, (1) by using a core in which the azithromycin is not substantially present on the exterior surface, the azithromycin being effectively encapsulated by the core excipients; or (2) by applying a protective barrier coat between the core and the enteric coating.

Cores wherein the azithromycin is not substantially present at the exterior surface of the core can be prepared using the thermal- and solvent-based processes previously described. In a preferred embodiment, the core is made using a melt-congeal process.

Alternately, wherein a barrier coat is used in combination with a reactive enteric coating, the azithromycin core is first coated with a barrier coat, and then is coated with the enteric coating. The barrier coat is located between the core and the enteric coating, effectively isolating the azithromycin-containing core from the coating materials. Examples of suitable barrier coat materials include long-chain alcohols, such as stearyl alcohol, cetyl alcohol, and polyethylene glycol; poloxamers; ethers, such as polyoxyethylene alkyl ethers; ether-substituted cellulosics, such as microcrystalline cellulose, hydroxypropyl cellulose, hydroxypropyl methyl cellulose, and ethylcellulose; sugars such as glucose, sucrose, xylitol, sorbitol, and maltitol; and salts such as sodium chloride, potassium chloride, lithium chloride, calcium chloride, magnesium chloride, sodium sulfate, potassium sulfate, sodium carbonate, magnesium sulfate, and potassium phosphate, and mixtures thereof. In some cases it may be desirable to add a nonreactive binder with such materials to improve the uniformity of the coating. Examples of such binders include maltodextrin, polydextrose, dextran, gelatin, hydroxyethyl cellulose, and hydroxypropyl cellulose. The barrier coat may comprise from about 1 wt % to about 100 wt %, preferably from about 2 wt % to about 50 wt %, of the weight of the uncoated azithromycin core.

In those embodiments where a barrier coat is used, a highly reactive enteric coating may also be used. Examples of suitable enteric coating materials for this embodiment include phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, cellulose acetate trimellitate, and mixtures thereof. In one embodiment, the coated multiparticulate comprises an azithromycin-containing core, a barrier coat, and an enteric coating, wherein the enteric coating is selected from the group consisting of phthalate-containing coatings and trimellitate-containing coatings.

The thickness of the enteric-release coating is adjusted to give the desired release property. In general, thicker coatings are more resistant to erosion and, consequently, yield a longer delay. Preferred coatings range from about 3 μm in thickness to about 3 mm in thickness. Preferably the coating comprises from about 5 wt % to about 200 wt % of the weight of the uncoated core. More preferably, the coating comprises from about 8 wt % to about 100 wt % of the weight of the uncoated core, even more preferably, the coating comprises from about 8 wt % to about 40 wt % of the weight of the uncoated core, and most preferably the coating comprises from about 15 wt % to about 30 wt % of the weight of the uncoated core.

In a preferred embodiment, enteric coated multiparticulates, of about 0.5 to 3.0 mm in diameter are coated with mixtures of polymers whose solubilities vary at different pH's. For example, preferred coatings comprise from about 9:1 to about 1:9 Eudragit-L®/Eudragit-S®, more preferably from 9:1 to 1:4 Eudragit-L®/Eudragit-S®. The coating may comprise from about 5% to about 200% of the weight of the uncoated core.

In another preferred embodiment, azithromycin multiparticulates, of about 0.01 to 0.5 mm in diameter, preferably 0.05 to 0.5 mm in diameter, are coated with one or more of enteric coating material comprising about 25% to about 200% of the weight of the uncoated azithromycin core.

### Coating Additives

Coating formulations often include additives to promote the desired release characteristics or to ease the application or improve the durability or stability of the coating to the core. Types of additives include plasticizers, pore formers, and glidants. Since such materials are part of the coating, their reactivity with azithromycin must also be considered.

Ideally, the coating additives have no reactive substituents. Examples of suitable coating additives which, in their pure forms have no reactive substituents, include plasticizers, such as mineral oils, petrolatum, lanolin alcohols, polyethylene glycol, polypropylene glycol, sorbitol and triethanol amine; pore formers, such as polyethylene glycol, polyvinyl pyrrolidone, polyethylene oxide, hydroxyethyl cellulose and hydroxypropylmethyl cellulose; and glidants, such as colloidal silicon dioxide, talc and cornstarch.

It is often desirable to use commercially available coating formulations that contain additives such as plasticizers in order to obtain uniformly reproducible coatings that are stable and durable. However, some of the additives in such formulations have substituents that can react to form azithromycin esters. Such materials are generally of a lower molecular weight, and so are highly mobile compared with higher molecular weight coating excipients. As a result, they may have high reaction rates with azithromycin to form azithromycin esters. Accordingly, if such a coating additive is used, it is preferred that the amount of azithromycin present on the exterior surface of the core be low and/or that a protective coating first be applied to the core to prevent contact of the coating additive with the azithromycin, thus keeping the amount of azithromycin esters at acceptable levels. Examples of materials that can be used for the protective coating include those listed above for use with highly reactive coating excipients.

### Coating Solvents

The coating can be formed using solvent-based coating processes and hot-melt coating processes. In solvent-based processes, the coating is made by first forming a solution or suspension comprising the solvent, the coating excipient and optional coating additives. The coating materials may be completely dissolved in the coating solvent, or only dispersed in the solvent as an emulsion or suspension or anywhere in between. The solvent used for the solution should be inert in the sense that it does not react with or degrade azithromycin, and be pharmaceutically acceptable. Preferably, to ensure low amounts of azithromycin esters form in the coating solution, the concentration of acid or ester substituents on the solvent is less than about 0.1 meq/g of solvent. In one aspect, the solvent is a liquid at room temperature. Preferably, the solvent is a volatile solvent. By “volatile solvent” is meant that the material has a boiling point of less than about 150° C. at ambient pressure, although small amounts of solvents with higher boiling points can be used and acceptable results still obtained.

Examples of solvents suitable for use in applying a coating to an azithromycin-containing core include alcohols, such as methanol, ethanol, isomers of propanol and isomers of butanol; ketones, such as acetone, methylethyl ketone and methyl isobutyl ketone; hydrocarbons, such as pentane, hexane, heptane, cyclohexane, methylcyclohexane, octane and mineral oil; ethers, such as methyl tert-butyl ether, ethyl ether and ethylene glycol monoethyl ether; chlorocarbons, such as chloroform, methylene dichloride and ethylene dichloride; tetrahydrofuran; dimethylsulfoxide; N-methylpyrrolidinone; acetonitrile; water; and mixtures thereof.

In another embodiment of the present invention, a suitable solvent is one in which azithromycin has a low solubility. Unless otherwise specified, the solubility of azithromycin in the solvent is measured at ambient temperature. The low solubility of azithromycin in such a solvent tends to retard the amount of azithromycin in the core that dissolves during the coating operation. This is desirable; since dissolved azithromycin is more reactive than solid azithromycin and reactions of dissolved azithromycin with materials in the coating formulation or in the core will be increased. In addition, amorphous azithromycin is generally more reactive than crystalline azithromycin. Dissolution of a portion of crystalline azithromycin during the coating process may re-solidify upon drying. However, this re-solidified azithromycin may be amorphous rather than crystalline and therefore may be more reactive. Preferably, the solubility of azithromycin in the solvent is less than about 10 mg/mL, more preferably less than about 5 mg/mL, and most preferably less than about 1 mg/mL.

Because azithromycin is a very hydrophilic compound, azithromycin has a low solubility in solvents that tend to be hydrophobic. Examples of suitable hydrophobic solvents include hydrocarbons, such as pentane, hexane, heptane, cyclohexane, methylcyclohexane, octane, mineral oil and the like.

The solubility of azithromycin in water is also highly pH-dependent, its solubility decreasing as pH increases. A preferred solvent for solvent-based application of coatings is water at a pH of 7 or greater. In such cases, the coating solution is often a suspension of the coating polymer in water, with additives to stabilize the suspension. Such coating formulations are often referred to in the pharmaceutical arts as a latex or pseudo-latex formulation. Water having a pH greater than neutral can be generated by dissolving a small amount of a base in the water, or by preparing a buffer solution that will precisely control the pH. Examples of bases that can be added to the water to raise the pH include hydroxides, such as sodium hydroxide, calcium hydroxide, ammonium hydroxide, choline hydroxide and potassium hydroxide; bicarbonates, such as sodium bicarbonate, potassium bicarbonate and ammonium bicarbonate; carbonates, such as ammonium carbonate and sodium carbonate; phosphates, such as sodium phosphate and potassium phosphate; borates, such as sodium borate; amines, such as tris(hydroxymethyl)-amino methane, ethanolamine, diethanolamine, N-methyl glucamine, glucosamine, ethylenediamine, cyclohexylamine, cyclopentylamine, diethylamine, isopropylamine and triethylamine; and proteins, such as gelatin. A particularly useful buffer is phosphate buffered saline (PBS) solution, which is an aqueous solution comprising 20 mM Na₂HPO₄, 466 mM KH₂PO₄, 87 mM NaCl and 0.2 mM KCl, adjusted to pH 7.

It will be appreciated by those of ordinary skill in the pharmaceutical arts that azithromycin cores can be coated using standard coating equipment, such as pan coaters (e.g., Hi-Coater available from Freund Corp. of Tokyo, Japan, Accela-Cota available from Manesty of Liverpool, U.K.), fluidized bed coaters (e.g., Würster coaters or top-spray coaters, available from Glatt Air Technologies, Inc. of Ramsey, N.J. and from Niro Pharma Systems of Bubendorf, Switzerland) and rotary granulators (e.g., CF-Granulator, available from Freund Corp). For example, when using a solvent-based process for forming the coating, a Würster fluidized-bed system is used. In this system, a cylindrical partition (the Würster column) is placed inside a conical product container in the apparatus. Air passes through a distribution plate located at the bottom of the product container to fluidize the cores, with the majority of the upward moving air passing through the Würster column. The cores are drawn into the Würster column, which is equipped with an atomizing nozzle that sprays the coating solution upward. The cores are coated as they pass through the Würster column, with the coating solvent being removed as the multiparticulates exit the column.

### Pharmaceutical Compositions

The coated multiparticulates of the invention may be mixed or blended with one or more pharmaceutically acceptable excipients, such as surfactants, conventional matrix materials, fillers, diluents, lubricants, preservatives, thickeners, anticaking agents, disintegrants, or binders, to form a suitable oral dosage form. Suitable dosage forms include tablets, capsules, sachets, oral powders for constitution and the like.

The term “tablet” is intended to embrace compressed tablets, coated tablets, and other forms known in the art. See for example, Remington's Pharmaceutical Sciences (19th Ed. 1995). Upon administration to the use environment, the tablet rapidly disintegrates, allowing the multiparticulates to be dispersed in the use environment.

In one embodiment, the tablet comprises multiparticulates mixed with a binder, disintegrants, or other excipients known in the art, and then formed into a tablet using compressive forces. Examples of binders include microcrystalline cellulose, starch, gelatin, polyvinyl pyrrolidinone, polyethylene glycol, and sugars such as sucrose, glucose, dextrose, and lactose. Examples of disintegrants include sodium starch glycolate, croscarmellose sodium, crospovidone, and sodium carboxymethyl cellulose. The tablet may also include an effervescent agent (acid-base combinations) that generates carbon dioxide when placed in the use environment. The carbon dioxide generated helps in disintegration of the tablet. Other excipients, such as those discussed above, may also be included in the tablet. The multiparticulates, binder, and other excipients used in the tablet may be granulated prior to formation of the tablet. Wet- or dry-granulation processes, well known in the art, may be used, provided the granulation process does not change the release profile of the multiparticulates. Alternatively, the materials may be formed into a tablet by direct compression. The compression forces used to form the tablet should be sufficiently high to provide a tablet with high strength, but not too high to damage the multiparticulates contained in the tablet. Generally, compression forces that result in tablets with a hardness of about 3 to about 10 kp are desired.

In another embodiment, the dosage form is in the form of a capsule. See Remington's Pharmaceutical Sciences (19th Ed. 1995). The term “capsule” is intended to embrace solid dosage forms in which the multiparticulates and optional excipients are enclosed in either a hard or soft, soluble container or shell.

The dosage form may also be in pills. The term “pill” is intended to embrace small, round solid dosage forms that comprise the multiparticulates mixed with a binder and other excipients as described above. Upon administration, the pill rapidly disintegrates, allowing the multiparticulates to be dispersed therein.

In another embodiment, the multiparticulate dosage form is in the form of a powder or granules comprising the multiparticulates and other excipients as described above, that is then suspended in a liquid dosing vehicle, including an aqueous dosing vehicle, prior to dosing. Such dosage forms may be prepared by several methods. In one method, the powder is placed into a container and an amount of a liquid, such as water, is added to the container. The container is then mixed, stirred, or shaken to suspend the dosage form in the water. In another method, the multiparticulates and dosing vehicle excipients are supplied in two or more separate packages. The dosing vehicle excipients are first dissolved or suspended in a liquid, such as water, and then the multiparticulates are added to the liquid vehicle solution. Alternatively, the dosing vehicle excipients and multiparticulates, in two or more individual packages, can be added to the container first, water added to the container, and the container mixed or stirred to form a suspension. Examples of suitable vehicles include water, beverages, and water mixed with other excipients to help form the dosage form, including surfactants, thickeners, suspending agents, and the like.

The present dosage forms provide a relative degree of improvement in toleration of administered azithromycin of at least 1.1 as compared to an equivalent immediate release dosage form. Preferably, the relative degree of improvement in toleration is at least about 1.25. More preferably, the relative improvement in toleration is at least about 1.5. Even more preferably, the relative improvement in toleration is at least about 2.0. Most preferably, the relative improvement in toleration is at least about 3.0. A “relative degree of improvement in toleration” is defined as the ratio of (1) the percentage adverse events arising from the administration of an immediate release control dosage form to (2) the percentage adverse events arising from the administration of a enteric coated multiparticulate dosage form of the present invention, where the immediate release control dosage form and the enteric coated multiparticulate dosage form contain the same amount of azithromycin. The immediate release control dosage form may be any conventional immediate release dosage form, such as Zithromax® tablets, capsules, or single-dose packets for oral suspension. For example, if an immediate release control dosage form provides a percentage adverse events arising from the administration of 20% while the enteric coated multiparticulate dosage form of the present invention provides a percentage adverse events arising from the administration of 10%, then the relative degree of improvement in toleration is 20%÷10% or 2.

Preferably, the present dosage forms also maintain a suitable bioavailability by not significantly reducing the azithromycin release rate and/or dissolution rate of administered azithromycin in the duodenum or distal to the duodenum. Typically, the present dosage forms have a bioavailability of at least 60%, more preferably at least 70%, even more preferably at least 80%, and most preferably at least 90% relative to the control composition.

The pharmaceutical dosage forms of the present invention are used to treat bacterial or protozoal infection(s) in a mammal by administering an effective amount of azithromycin to said mammal. The term “effective amount of azithromycin” means the amount of azithromycin which, when administered, according to the present invention, prevents the onset of, alleviates the symptoms of, stops the progression of, or eliminates a bacterial or protozoal infection in a mammal. The term “mammal” is an individual animal that is a member of the taxonomic class Mammalia. The class Mammalia includes, for example, humans, monkeys, chimpanzees, gorillas, cattle, swine, horses, sheep, dogs, cats, mice and rats. In the present invention, the preferred mammal is a human.

For adult humans, and for pediatric humans weighing more than 30 kg, the amount of azithromycin administered in a dose is typically between about 250 mgA and about 7 gA. The term “gA” refers to grams of active azithromycin, meaning the non-salt, non-hydrated azithromycin macrolide molecule having a molecular weight of 749 g/mol. Preferably, for adult humans, and for pediatric humans above 30 kg in weight, the dose form contains between about 1.5 to about 4 gA, more preferably about 1.5 to about 3 gA, and most preferably about 1.8 to about 2.2 gA. For pediatric humans weighing 30 kg, or less, the azithromycin dose is typically scaled, according to the weight of the patient, and contains about 30 to about 90 mgA/kg of patient body weight, preferably about 45 to about 75 mgA/kg, and more preferably about 60 mgA/kg. The azithromycin may be administered using a single-dose therapy or in multiple-dose therapy (e.g., administering more than one dose in a single day or administering one or more doses over a course of 2-5 days or more). A daily dosage can be administered from 1 to 4 times daily in equal doses. Preferably, the azithromycin is administered in one dose per day. More preferably, a full course of azithromycin therapy consist of one single dose of azithromycin.

For animal/veterinary applications, the amount can, of course, be adjusted to be outside these limits depending, for example, on the size of the animal subject being treated.

### EXEMPLIFICATION

The present invention will be further illustrated by means of the following examples. In the examples that follow, the following definitions are employed:

Quantities in percent (%) means percent by weight based on total weight, unless otherwise indicated.

Lutrol® F127 NF (hereinafter referred to as “Lutrol®”) and Pluronic® F127 (hereinafter referred to as “Pluronic®”), which are also known as Poloxamer 407 NF, are polyoxypropylene-polyoxyethylene block copolymers having a molecular weight, calculated on the OH value, of 9,840 to 14,600 g/mol and having a general structure of

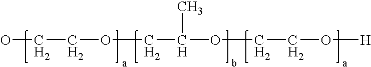

wherein a is about 101 and b is about 56, obtained from BASF Corporation, Mount Olive, N.J. Lutrol® is the pharmaceutical equivalent of Pluronic®.

Compritol® 888 ATO (hereinafter referred to as “Compritol®”), which is composed of a mixture of glyceryl mono-, di- and tribehenates, the diester fraction being predominant, is synthesized by esterification of glycerol by behenic acid (C22 fatty acid) and then atomized by spray-cooling, was obtained from GATTEFOSSÉ Corporation, Saint Priest, Cedex, France.

### Example 1

This example illustrates a process for making multiparticulates for use in making delayed-release dosage forms designed to release azithromycin predominantly below the duodenum. The process comprised (1) preparing uncoated azithromycin multiparticulate cores; (2) applying a first, sustained-release coating over the cores; and (3) applying a second, enteric (pH-sensitive, delayed-release) coating over the first coat.

Multiparticulate cores containing drug were prepared using a fluid bed processor with rotor insert (Model GPCG-5). The rotor bowl was initially charged with 2,500 g of azithromycin and plasticized hydroxypropyl methylcellulose (Opadry®, Colorcon, West Point, Pa.) binder solution (10% solids concentration) was sprayed into the rotating bed until an average core granule size of about 250 μm was achieved. Next, a plasticized ethylcellulose (Surelease™) coating suspension diluted to 15 wt % solids was sprayed onto the core particles. A first batch of coated particles was made with a total 30 wt % coating and 60 wt % core. A second batch was then made with a 40 wt % coating and 60 wt % core. Lastly, both batches of multiparticulate were coated with an enteric coating in a fluid bed rotor processor (Glatt Model GPCG-1) until a desired coating end point was achieved. The enteric coating was a suspension containing 12.3% methacrylic acid copolymers (Eudragit™ L 30 D-55), 6.2% talc, 1.5% triethyl citrate and 80% water. For the first batch that had been coated with a 40% Surelease™ coat, a 20% enteric coat was applied. For the second batch that had been coated with a 30% Surelease™ coat, a 33.7 wt % enteric coat was applied. The final product was enteric coated multiparticulate with particles having an average size of about 300 μm.

### Example 2

This example illustrates a process for making multiparticulates for use in making delayed-release dosage forms designed to release azithromycin predominantly below the duodenum. The process comprises (1) preparing uncoated azithromycin multiparticulate cores; (2) applying a first, sustained-release diffusion barrier coating over the cores; and (3) applying a second, enteric (pH-sensitive, delayed release) coating over the first coat.

Azithromycin-containing multiparticulate cores are prepared by blending azithromycin compound with microcrystalline cellulose (Avicel™ PH101, FMC Corp., Philadelphia, Pa.) in relative amounts of 95:5 (w/w), wet massing the blend in a Hobart mixer with water equivalent to approximately 27 wt % of the weight of the blend, extruding the wet mass through a perforated plate (Luwa EXKS-1 extruder, Fuji Paudal Co., Osaka Japan), spheronizing the extrudate (Luwa QJ-230 marumerizer, Fuji Paudal Co.) and drying the final cores which are about 1 mm diameter.

Next, a Würster bottom spray fluid bed processor (Glatt GPCG-1) is used to coat the uncoated azithromycin-containing multiparticulate with a diffusion barrier coating. A plasticized ethylcellulose (Surelease™) coating suspension diluted to 15% solids is sprayed onto the core particles. Typically, a 5% to 20% diffusion barrier coating is applied. The amount of barrier coating applied determines the rate of azithromycin release from the uncoated core.

Lastly, a Würster bottom spray fluid bed processor (Glatt GPCG-1) is used to apply an enteric coating over the diffusion barrier coated particles. Typical enteric coating levels are 25% to 50%. The enteric coating is a suspension containing 12.3% methacrylic acid copolymers (Eudragit™ L 30 D-55), 6.2% talc, 1.5% triethyl citrate and 80% water.

Because the delayed release coating is soluble in environments where the pH is greater than 5.5, the multiparticulates thus prepared release azithromycin from the barrier coated particle cores below the stomach where the pH is greater than 5.5, and the particle cores do so in a sustained manner that delivers azithromycin predominantly below the duodenum.

### Example 3

This example illustrates a process for making multiparticulates for use in making delayed-release dosage forms designed to release azithromycin predominantly below the duodenum. The process comprises (1) preparing uncoated azithromycin multiparticulate cores; (2) applying a protective coat over the core particles; and (3) applying a second, enteric (pH-sensitive, delayed release) coating over the first coat.

Multiparticulate cores containing drug are prepared using a fluid bed processor with rotor insert (Model GPCG-1). The rotor bowl is initially charged with 400 g of azithromycin drug and a binder solution containing 5 wt % poly(ethyl acrylate, methyl acrylate) (Eudragit NE-30-D), 5 wt % plasticized hydroxypropyl methylcellulose (Opadry™) and 90% water is sprayed into the rotating bed until an average core granule size of about 250 μm was achieved.

Onto the uncoated core particles in the same fluid bed processor with rotor insert, a binder solution containing 5 wt % plasticized hydroxypropyl methylcellulose (Opadry™) solution is sprayed until a coating of 10 wt % is applied. This intermediate coating enhances the adhesion to the core particles of the final enteric coating.

An enteric coating (typically 15 wt % to 50 wt %) is applied using the same fluid bed processor as above. The enteric coating is a suspension containing 12.3% methacrylic acid copolymers (Eudragit™ L 30 D-55), 6.2% talc, 1.5% triethyl Citrate and 80% water. The final product is an enteric coated multiparticulate with particles having an average size of about 300 μm.

### Method for Identifying Suitable Enteric Coatings Having Suitably Low Reactivity with Azithromycin

The reactivity of the materials useful for forming enteric coatings listed in Table 1 with azithromycin was determined as follows. Mixtures 50/50 (w/w) of azithromycin and of various materials, specifically cellulose acetate phthalate (CAP), hydroxypropyl cellulose acetate succinate (HPMCAS), cellulose acetate (CA), cellulose acetate trimellitatec (CAT), and triacetin, were prepared by adding equal weights of azithromycin and the material to a mortar and mixing with a spatula. The mixture was then placed in a controlled atmosphere oven at 50° C. and 20% RH for the storage times listed in Table 1.

Azithromycin esters were identified in each of the mixtures by LC/MS detection. Samples were prepared by extraction with methanol at a concentration of 1.25 mg azithromycin/mL and sonication for 15 minutes. The sample solutions were then filtered with a 0.45 μm nylon syringe filter. The sample solutions were then analyzed by HPLC using a Hypersil BDS C18 4.6 mm×250 mm (5 μm) HPLC column on a Hewlett Packard HP1100 liquid chromatograph. The mobile phase employed for sample elution was a gradient of isopropyl alcohol and 25 mM ammonium acetate buffer (pH approximately 7) as follows: initial conditions of 50/50 (v/v) isopropyl alcohol/ammonium acetate; the isopropyl alcohol percentage was then increased to 100% over 30 minutes and held at 100% for an additional 15 minutes. The flow rate was 0.80 mL/min. The method used a 75 μL injection volume and a 43° C. column temperature. A Finnigan LCQ Classic mass spectrometer was used for detection. The Atmospheric Pressure Chemical Ionization (APCI) source was used in a positive ion mode with a selective ion-monitoring method. Azithromycin ester values were calculated from the MS peak areas based on an external azithromycin standard. The azithromycin ester values were reported as percentage of the total azithromycin in the sample. The results of this analysis are shown below as are the measured rates of ester formation (Rₑ) at 50° C.

| Screening | | Storage | Concentration of | Rₑ |
|---|---|---|---|---|
| Example | | Time | Azithromycin | at 50° C. |
| No. | Material | (days) | Esters (wt %) | (wt %/day) |
| 1 | CAP | 11 | 0.012 | 1.1 × 10⁻³ |
| 2 | HPMCAS | 35 | 0.004 | 1.1 × 10⁻⁴ |
| 3 | CA | 35 | 0.003 | 8.6 × 10⁻⁵ |
| 4 | CAT | 10 | 0.09 | 9.0 × 10⁻³ |
| 5 | Triacetin | 10 | 0.65 | 6.5 × 10⁻² |

Using the Rₑ values for coatings set forth above for formation of compositions with low concentrations of azithromycin esters, the maximum allowable rates of ester formation (Rₑₘₐₓ) at 50° C. to achieve the desired low concentration of esters were calculated. The results of these calculations are given below.

| Maximum Concentration | | |
|---|---|---|
| of Azithromycin | | |
| Esters in the | | Rₑₘₐₓ |
| Composition | Rₑ Values | at 50° C. |
| (wt %) | for Coatings | (wt %/day) |
| <5 | ≦1.8 × 10⁸•e^{-7070(T + 273)} | 5.6 × 10⁻² |
| <1 | ≦3.6 × 10⁷•e^{-7070(T + 273)} | 1.1 × 10⁻² |
| <0.5 | ≦1.8 × 10⁷•e^{-7070(T + 273)} | 5.6 × 10⁻³ |
| <0.1 | ≦3.6 × 10⁶•e^{-7070(T + 273)} | 1.1 × 10⁻³ |

Comparison of these calculated maximum rates of ester formation with those measured above using mixtures of azithromycin and enteric coating materials, show that four of the coating materials, with the exception of triacetin, would be suitable to obtain compositions with the preferred range of less than 5 wt % esters. The higher rate of ester formation with triacetin indicates that this excipient should only be used with a protective coating around the core or with a core having a low concentration of azithromycin on its exterior surface to obtain compositions with less than 5 wt % azithromycin esters. To obtain multiparticulates with less than 1 wt % azithromycin esters, use of a protective coating or a core having a low concentration of azithromycin on its exterior surface is also needed for cellulose acetate trimellitate. Similarly, to obtain multiparticulates with less than 0.1 wt % azithromycin esters, a protective coating or a core having a low concentration of azithromycin on its exterior surface is needed with cellulose acetate phthalate. The data also show that the rate of ester formation for HPMCAS (No. 2) and CA (No. 3) are well below the calculated maximum values for obtaining compositions with low concentrations of azithromycin esters. Thus, these excipients can be used as coating materials without the need for a protective layer or cores with low concentrations of azithromycin on the exterior surface.

### Preparation of Uncoated Azithromycin Multiparticulates

Uncoated multiparticulates UM1 comprising 50 wt % azithromycin, 40 wt % stearyl alcohol and 10 wt % of a poloxamer 407 (PLURONIC F127, BASF Corp. of Parsippany, N.J.) were prepared as follows. Stearyl alcohol (1600 g) and 400 g of poloxamer 407 were placed in a container and heated to about 100° C. on a hot plate. Next, 2000 g of azithromycin dihydrate was added to the melt and mixed by hand using a spatula for about 15 minutes, resulting in a feed suspension of the azithromycin in the molten components. The feed suspension was pumped at a rate of about 250 g/min using a gear pump (Zenith Pumps, Sanford, N.C.) to the center of a 10-cm diameter spinning-disk atomizer to form azithromycin multiparticulates. The spinning disk atomizer, which was custom made, consists of a bowel-shaped stainless steel disk of 10.1 cm (4 inches) in diameter. The surface of the disk is heated with a thin film heater beneath the disk to about 90° C. That disk is mounted on a motor that drives the disk of up to approximately 10,000 RPM. A suitable commercial equivalent, to this spinning disk atomizer, is the FX1 100-mm rotary atomizer manufactured by Niro A/S (Soeborg, Denmark). The surface of the spinning disk atomizer was maintained at 100° C., and the disk was rotated at 3200 rpm, while forming the azithromycin multiparticulates. The particles formed by the spinning-disk atomizer were congealed in ambient air and collected. The azithromycin multiparticulates, prepared by this method, had a mean particle size of about 180 μm determined using a scanning electron microscope.

Uncoated multiparticulates UM2 comprising 50 wt % azithromycin and 50 wt % stearyl alcohol were formed using the procedures used to form UM1 with the following exceptions. The feed was melted at about 85° C. and consisted of 750 g of stearyl alcohol and 750 g of azithromycin dihydrate. The disk speed was 4800 rpm and its temperature was about 95° C. The resulting particles had a mean particle diameter of about 250 μm.

Uncoated multiparticulates UM3 comprising 70 wt % azithromycin and 30 wt % stearyl alcohol were formed using the procedures used to form UM1 with the following exceptions. The feed was melted at about 100° C. and consisted of 121 g of stearyl alcohol and 282 g of azithromycin dihydrate. The disk speed was 6700 rpm and its temperature was about 95° C. The resulting particles had a mean particle diameter of about 180 μm.

Uncoated multiparticulates UM4 comprising 50 wt % azithromycin in a carrier of 46 wt % glyceryl mono-, di-, and tri-behenates (COMPRITOL 888 from Gattefosse of France) and 4 wt % of a poloxamer (LUTROL F127 from BASF of Mount Olive, N.J.) were prepared using the following procedure. A mixture of 2.5 kg] azithromycin dihydrate, 2.3 kg of the COMPRITOL and 0.2 kg of the LUTROL were blended in a V-blender (Blend Master, Patterson-Kelley Co., East Straudsburg, Pa.) for 20 minutes. This blend was then milled using a Fitzpatrick M5A mill (The Fitzpatrick Company, Elmhurst, Ill.) at 3000 rpm, knives forward using a 0.065-inch screen. The milled blend was then placed back into a V-blender for an additional 20 minutes. Three batches of this blended material were then combined to form a preblend feed. The preblend feed was delivered to a B&P 19-mm twin-screw extruder (MP19-TC with a 25 L/D ratio purchased from B & P Process Equipment and Systems, LLC, Saginaw, Mich.) at a rate of 140 g/min. The extruder was set such that it produced a molten feed suspension of the azithromycin in the COMPRITOL/LUTROL at a temperature of about 90° C. The feed suspension was then delivered to the spinning-disk atomizer, used for UM1. The spinning-disk atomizer was enclosed in a plastic bag of approximately 8 feet in diameter to allow congealing and to capture multiparticulates formed by the atomizer. Air was introduced from a port underneath the disk to provide cooling of the multiparticulates upon congealing and to inflate the bag to its extended size and shape. To form the multiparticulates, the spinning-disk atomizer was rotating at 5500 rpm, and the surface was maintained at about 90° C. The mean particle size of the resulting multiparticulates was determined to be about 210. The multiparticulates were then post-treated by placing them in a shallow tray at a depth of about 2 cm. This tray was then placed in a controlled atmosphere oven at 40° C. and 75% RH for 5 days.

Uncoated multiparticulates UM5 comprising 50 wt % azithromycin in a carrier of 46 wt % glyceryl mono-, di-, and tri-behenates (COMPRITOL 888 from Gattefosse of France) and 4 wt % of a poloxamer (LUTROL F127 from BASF of Mount Olive, N.J.) were prepared using procedures similar to those described for uncoated multiparticulates UM4 except that a Leistritz 27-mm extruder was used to form the molten mixture.

### Rate of Drug Release from Azithromycin Multiparticulates

The rates of release of azithromycin from the uncoated azithromycin multiparticulates UM1, UM2, UM3, and UM4 were determined. For samples of UM1, UM2, and UM3, the following dissolution procedure was used. A 750 mg sample of an uncoated multiparticulate was wetted with 10 mL of the a 0.01 N HCl (pH 2) simulated gastric buffer (GB) maintained at 37.0±0.5° C. and then placed into a USP Type 2 dissoette flask equipped with Teflon-coated paddles rotating at 50 rpm. The flask contained an additional 750 mL of the simulated GB. A 3 mL sample of the fluid in the flask was then collected at the elapse of the times, shown in Table 4, following the addition of the multiparticulate sample to the flask. The sample was filtered using a 0.45-μm syringe filter prior to analyzing via HPLC (Hewlett Packard 1100, Waters Symmetry C₈ column, 45:30:25 acetonitrile:methanol:25 mM KH₂PO₄ buffer at 1.0 mL/min, absorbance measured at 210 nm with a diode array spectrophotometer).

A similar dissolution protocol was used to test the rate of release of azithromycin from a sample of the UM4 multiparticulates. A dosing vehicle was prepared by dissolving 21.8 g of a mixture consisting of 98.2 wt % sucrose, 0.2 wt % hydroxypropyl cellulose, 0.2 wt % xanthan gum, 0.5 wt % colloidal SiO₂, 0.4 wt % cherry favoring, and 0.6 wt % banana flavoring in a pH 3.0 citrate buffer. A sample of multiparticulates containing 500 mgA of azithromycin was then placed in a vial and 60 mL of the dosing vehicle warmed to 37° C. was added to the multiparticulates. The vial was mixed for 30 seconds and the suspension of multiparticulates was then added to 690 mL of a 0.01 M HCl simulated GB dissolution medium. The vial was rinsed with two 20-mL aliquots of 0.01 N HCl, which were also added to the simulated GB. The total volume of the simulated GB was 750 mL.

The results of these dissolution tests, provided below, show that essentially all azithromycin was released from all of the uncoated azithromycin multiparticulates between 2.5 to 60 minutes.

| | | Azithromycin |
|---|---|---|
| | Time | Released |
| Example | (min) | (%) |
| UM1 | 2.5 | 97 |
| | 5 | 101 |
| | 7.5 | 100 |
| | 10 | 100 |
| | 15 | 100 |
| | 30 | 100 |
| | 60 | 100 |
| UM2 | 2.5 | 41 |
| | 5 | 65 |
| | 7.5 | 80 |
| | 10 | 88 |
| | 15 | 95 |
| | 30 | 101 |
| | 60 | 100 |
| UM3 | 2.5 | 51 |
| | 5 | 82 |
| | 7.5 | 95 |
| | 10 | 99 |
| | 15 | 102 |
| | 30 | 100 |
| | 60 | 100 |
| UM4 | 15 | 56 |
| | 30 | 84 |
| | 60 | 95 |
| | 120 | 95 |
| | 180 | 95 |

### Preparation of Enterically-Coated Azithromycin Multiparticulates Coated Multiparticulates CM1. CM2. CM3 and CM4

Coated multiparticulates (CM1, CM2, CM3 and CM4) were prepared by coating samples of azithromycin multiparticulates UM1 with an enteric polymer to delay release of the azithromycin as follows. A spray solution was prepared by dissolving 8 wt % of the HG grade of hydroxypropyl methyl cellulose acetate succinate (HPMCAS-HG from Shin Etsu), in 87.4 wt % acetone and 4.6 wt % water. The multiparticulates were fluidized in a Glatt GPCG-1 fluidized bed coater (Glatt Air Technologies, Ramsey, N.J.) equipped with a Würster column set at 13 mm. Fluidizing gas (nitrogen) was circulated through the bed at a rate of 1100 to 1200 L/min at an inlet temperature of 36° C. and a bed temperature of 28 to 29° C. The spray solution was introduced to the bed through a two-fluid nozzle at a rate of 7 to 12 g/min using nitrogen with an atomization pressure of 2.3 bar. Samples of the coated multiparticulates were collected at 84 minutes for CM1 multiparticulates (coating amount 11 wt %), 139 minutes for CM2 multiparticulates (coating amount 18 wt %), 237 minutes for CM3 multiparticulates (coating amount 28 wt %), and 293 minutes for CM4 multiparticulates (coating amount 33 wt %), resulting in multiparticulates with the specified coatings. The coating amount was calculated as the weight of coating material applied divided by the final weight of the coated core multiplied by 100%.

Coated multiparticulates CM5 were prepared from UM1 multiparticulates by coating the multiparticulates with HPMCAS-HG using the method of CM1 with the following exceptions. The inlet fluidizing gas temperature was set at 41° C. and the atomization pressure was set at 2 bar. The multiparticulates were coated for 120 minutes, resulting in a coating amount of 16.1 wt %.

Coated multiparticulates CM6, CM7 and CM8 were prepared from UM2 multiparticulates by coating the multiparticulates with HPMCAS-HG using the method of CM1. Samples of the coated multiparticulates were collected at 91.5 for CM6 multiparticulates (coating amount 8.7 wt %), 169 minutes for CM7 multiparticulates (coating amount 16.8 wt %), and 250 minutes for CM8 multiparticulates (coating amount 25.2 wt %), resulting in multiparticulates with the specified coatings.

Coated multiparticulates CM9 were prepared from UM3 multiparticulates by coating the multiparticulates with HPMCAS-HG using the method of CM1. The multiparticulates were coated for 105 minutes, resulting in a coating amount of 10.6 wt %.

Coated multiparticulates CM10 were prepared from UM4 multiparticulates by coating the multiparticulates with an enteric polymer to delay release of the azithromycin as follows. A latex spray solution was prepared comprising 16 wt % of EUDRAGIT L30D-55 (a 1:1 copolymer of methacrylic acid and ethyl acrylate from Röhm GmbH), 1.6 wt % triethyl citrate and 82.4 wt % water. The multiparticulates were fluidized in a Glatt GPCG-1 fluidized bed coater equipped with a Würster column set at 15 mm. Fluidizing gas (air) was circulated through the bed at a rate of 850 to 960 L/min at an inlet temperature of 39° to 41° C. and a bed temperature of 29° C. The spray solution was introduced to the bed through a two-fluid nozzle at a rate of 4.8 to 6.0 g/min using air with an atomization pressure of 2.1 bar. The multiparticulates were coated for about 190 minutes, resulting in multiparticulates with an average coating weight of about 23%. Following application of the coating, the multiparticulates were dried in the fluidized bed for 15 minutes at 29-32° C. The coated multiparticulates were then dried in a convection oven at 30° C. for 6 hours.

Coated multiparticulates CM11 were prepared from UM5 multiparticulates by coating the multiparticulates with an enteric polymer using the process of CM10. The resulting coated multiparticulates CM11 had a coating weight of 24.5 wt % based on the weight of the coated multiparticulates.

### Rates of Drug Release from Coated Multiparticulates

The rates of release of azithromycin from coated multiparticulates CM1, CM2, CM3, CM4, CM6, CM7 and CM8 were determined using the process previously described for dissolution testing uncoated azithromycin multiparticulate samples. The results of these dissolution tests, provided below, show that the application of an enteric coating to the multiparticulates delayed the release of the azithromycin. The data also show that as the greater the amount of coating applied to the multiparticulates, the slower the rate of azithromycin release.

| | | Azithromycin |
|---|---|---|
| | Time | Released |
| Multiparticulate | (min) | (%) |
| CM1 | 0 | 0 |
| | 5 | 1.9 |
| | 10 | 13.2 |
| | 15 | 20.4 |
| | 30 | 69.4 |
| | 45 | 91.0 |
| | 60 | 94.1 |
| CM2 | 0 | 0 |
| | 5 | 3.9 |
| | 10 | 9.6 |
| | 15 | 14.7 |
| | 30 | 31.3 |
| | 45 | 47.7 |
| | 60 | 61.5 |
| CM3 | 0 | 0 |
| | 5 | 4.3 |
| | 10 | 6.9 |
| | 15 | 9.1 |
| | 30 | 13.3 |
| | 45 | 18.1 |
| | 60 | 25.0 |
| CM4 | 0 | 0 |
| | 5 | 3.7 |
| | 10 | 2.9 |
| | 15 | 3.1 |
| | 30 | 3.9 |
| | 45 | 5.0 |
| | 60 | 4.9 |
| CM6 | 0 | 0 |
| | 5 | 15 |
| | 10 | 33 |
| | 15 | 46 |
| | 30 | 72 |
| | 60 | 90 |
| | 75 | 100 |
| CM7 | 0 | 0 |
| | 5 | 3 |
| | 10 | 10 |
| | 15 | 16 |
| | 30 | 35 |
| | 60 | 63 |
| | 75 | 93 |
| CM8 | 0 | 0 |
| | 5 | 1 |
| | 10 | 3 |
| | 15 | 5 |
| | 30 | 7 |
| | 60 | 34 |
| | 75 | 81 |

### Rate of Drug Release

The rate of release of azithromycin from coated multiparticulates CM10 was determined in a GB-IB transfer test, using the following procedure, conducted in a USP Type 2 dissolution flask equipped with Teflon-coated paddles, with stirring at 50 rpm and at 37° C. The same dosing vehicle used for UM4 was prepared and the coated multiparticulates CM10 were added to 750 mL of a 0.01 N HCl simulated GB dissolution medium. The vial was rinsed with two 20 mL aliquots of 0.01 N HCl, which were also added to the simulated GB. After 60 minutes, 250 mL of a 0.2 M KH₂PO₄ buffer solution at pH 7.2 was added to the simulated GB, so that the resulting dissolution medium simulated an IB at a pH of about 6.8.

A 3 mL sample of the fluid in the dissolution flask was collected after the elapse of the times reported in Table 15 following addition of the multiparticulates to the flask. The samples were filtered using a 0.45-μm syringe filter prior to analyzing via HPLC (Hewlett Packard 1100, Waters Symmetry C₈ column, 45:30:25 acetonitrile:methanol:25 mM KH₂PO₄ buffer at 1.0 mL/min, absorbance measured at 210 nm with a diode array spectrophotometer).

The results of this dissolution test, provided below, show that the coated multiparticulates provided enteric protection, with only 10 wt % of the azithromycin being released after 1 hour in simulated GB. Following transfer to the simulated IB, the multiparticulates rapidly released the azithromycin, with 90% being released after 3 hours.

| Time | Azithromycin | Time | Azithromycin |
|---|---|---|---|
| (hrs) | Released (%) | (hrs) | Released (%) |
| 0.25 | 2 | 1.5 | 62 |
| 0.5 | 4 | 2.0 | 78 |
| 1.0 | 10 | 3.0 | 90 |
| 1.25 | 49 | 4.0 | 93 |

### Rates of Ester Formation

Coated multiparticulates CM3, CM8 and CM9 were stored, respectively for 329, 316 and 315 days, at ambient temperature (about 22° C.) and ambient humidity (about 40% RH) and then analyzed for azithromycin esters by LC/MS detection. Samples were prepared by extraction with methanol at a concentration of 1.25 mg azithromycin/mL and sonication for 15 minutes. The sample solutions were then filtered with a 0.45 μm nylon syringe filter. The sample solutions were then analyzed by HPLC using a Hypersil BDS C18 4.6 mm×250 mm (5 μm) HPLC column on a Hewlett Packard HP1100 liquid chromatograph. The mobile phase employed for sample elution was a gradient of isopropyl alcohol and 25 mm ammonium acetate buffer (pH approximately 7) as follows: initial conditions of 50/50 (v/v) isopropyl alcohol/ammonium acetate; the isopropyl alcohol percentage was then increased to 100% over 30 minutes and held at 100% for an additional 15 minutes. The flow rate was 0.80 mL/min. A 75 μL injection volume and a 43° C. column temperature were used.

A Finnigan LCQ Classic mass spectrometer was used for detection. The APCI source was used in positive-ion mode with a selective ion-monitoring method. Azithromycin ester values were calculated from the MS peak areas based on an external azithromycin standard. The azithromycin ester values were reported as a percentage of the total azithromycin in the sample. The results of these tests showed that the concentration of azithromycin esters in these samples was less than 0.001 wt %. The reaction rate at 22° C. for the formation of azithromycin esters was also determined to be very low, specifically less than 3.0×10⁻⁶ wt %/day, which is below the maximum allowable value for a composition with less than 5 wt % azithromycin esters at 22° C. (calculated using the equation Rₑ≦1.8×10⁸·e^{-7070/(T+273)} to be <7×10⁻³ wt %/day).

The coated multiparticulates CM5 were stored in foil/foil pouches at 40° C. and 75% RH for 21 days and then stored at ambient temperature and humidity for 314 days. After storage, the coated multiparticulates were analyzed for azithromycin esters. The results of this analysis showed that the coated multiparticulates had an azithromycin ester concentration of 0.004 wt %, corresponding to a rate of ester formation of 1.3×10⁻⁵ wt %/day, or well below the maximum allowable reaction rate under these storage conditions for achieving compositions with less than 5 wt % azithromycin esters.

Coated multiparticulates CM 18 were stored at 40° C. and 75% RH for 6 weeks and then analyzed for azithromycin esters. None were detected in the multiparticulates.

### Pharmacokinetics Clinical Study

The in vivo pharmacokinetics of a 2000 mgA dose of coated multiparticulates CM11, in an oral dosing vehicle, were evaluated in 15 fasting, healthy human subjects in a randomized two-way crossover study. The oral dosing vehicle was prepared by dissolving 21.8 g of a mixture consisting of 98.2 wt % sucrose, 0.17 wt % hydroxypropyl cellulose, 0.17 wt % xanthan gum, 0.5 wt % colloidal SiO₂, 0.35 wt % cherry favoring, and 0.583 wt % banana flavoring in a pH 3.0 citrate buffer.

As a control, each member of each group tested received two single dose packets of azithromycin dihydrate for oral suspension (Zithromax®, Pfizer Inc., New York, N.Y.) wherein each dose contains 1048 mg azithromycin dihydrate as well as the inactive ingredients colloidal silicon dioxide, anhydrous tribasic sodium phosphate, artificial banana and cherry flavors and sucrose.

On Day 1, 7 subjects each received the 2 gA CM11 dosage form and 8 subjects each received 2 gA of the Control dosage form. Both dosage forms were administered by adding each to a bottle containing 120 mL of distilled water. Each subject drank the contents and the bottle was then refilled with 120 mL of distilled water, which the subject also drank. Azithromycin concentrations in each subject's blood serum were measured for 96 hours following administration of each dosage form.

All subjects were orally dosed after an overnight fast. All subjects were then required to refrain from lying down, eating and drinking beverages other than water during the first 4 hours after dosing.

Blood samples (5 mL each) were withdrawn from the subjects' veins prior to dosing, and at 0.5, 1, 2, 3, 4, 6, 8, 12, 16, 24, 36, 48, 72 and 96 hr post-dosing. Serum azithromycin concentrations were determined using the high performance liquid chromatography assay described in Shepard et al., J. Chromatography. 565:321-337 (1991). Total systemic exposure to azithromycin was determined by measuring the area under the curve (AUC) for each subject in the group and then by calculating a mean AUC for the group. Cmax is the highest serum azithromycin concentration achieved in a subject. Tmax is the time at which Cmax is achieved.

On Day 15, the subjects who received Control dosage form on Day 1 were dosed with the CM11 dosage form, while the subjects who received the CM11 dosage form on Day 1 were dosed with the Control dosage form. The results of this study are provided below.

| | | Tₘₐₓ | AUC_{0-Tlast}, |
|---|---|---|---|
| | Cₘₐₓ (μg/mL) | (hr) | (μg • hr/mL) |
| | Adjusted | Adjusted | Adjusted |
| | Geometric | Arithmetic | Geometric |
| Dosage Form | Means | Means | Means |
| CM11 | 1.04 | 4.0 | 15.9 |
| Control | 2.05 | 1.2 | 18.9 |
| Ratio (%) CM11/Control | 50.8 | | 83.9 |
| Difference CM11-Control | | 2.79 | |

These results show that coated multiparticulates CM11 provided a relative bioavailability of about 84% in comparison to the Immediate Release Control. Also, the time to achieve the maximum serum concentration was longer for the coated azithromycin multiparticulate dosage form than for the immediate release control dosage form.

The lower observed Cₘₐₓ for the CM11 coated multiparticulates also resulted in reduced incidence of gastrointestinal side effects. Subjects were queried regarding adverse events (AEs) during each treatment period at 1, 2, 4, 8, 12, 16, 24; 36, 48, 72, and 96 hours following dosing. Of the events that were considered to be moderate in intensity, only the diarrhea, nausea, and vomiting that occurred following the single dose of the control were considered to be treatment related.

| | Treatment | |
|---|---|---|
| | Group (n) | |
| Adverse | CM11 | Control |
| Events | (n = 16) | (n = 15) |
| Abdominal | 1 | 0 |
| Pain | | |
| Diarrhea | 1 | 1 |
| Nausea | 0 | 3 |
| Vomiting | 0 | 3 |

We claim:

1. A pharmaceutical composition comprising multiparticulates, wherein said multiparticulates comprise an azithromycin core and an enteric coating disposed upon said azithromycin core.

2. The pharmaceutical composition of claim 1 wherein said enteric coating has a thickness of between about 3 μm to about 3 mm.

3. The pharmaceutical composition of claim 1 wherein there is a concentration of azithromycin esters in said composition is less than about 5 wt % relative to the total weight of azithromycin originally present in the composition.

4. The pharmaceutical composition of claim 1 wherein said azithromycin is substantially in the form of the crystalline dihydrate.

5. The pharmaceutical composition of any of claims 1-4 wherein said enteric coating comprises at least one material selected from the group consisting of polyacrylamides, acid phthalates of carbohydrates, amylose acetate phthalate, cellulose acetate phthalate, cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, cellulose acetate trimellitate, hydroxypropyl methylcellulose acetate succinate, cellulose acetate succinate, carboxymethyl cellulose, carboxyethyl cellulose, carboxymethyl ethyl cellulose, styrene and maleic acid copolymers, polyacrylic acid derivative, polymethacrylic acid and esters thereof, poly acrylic methacrylic acid copolymers, shellac, vinyl acetate and crotonic acid copolymers, and mixtures thereof.

6. The pharmaceutical composition of any of claims 1-4 wherein said enteric coating comprises at least one material selected from the group consisting of carboxymethyl cellulose, carboxyethyl cellulose, carboxymethyl ethyl cellulose, styrene and maleic acid copolymers, polyacrylic acid, polymethacrylic acid, polyacrylic and methacrylic acid copolymers, crotonic acid copolymers, hydroxypropylmethyl cellulose acetate succinate, and mixtures thereof.

7. The pharmaceutical composition of any of claims 1-4 wherein said enteric coating comprises a mixture of (i) a copolymer of methacrylic acid and ethyl acrylate and (ii) triethyl citrate.

8. The pharmaceutical composition of any of claims 1-4 wherein said azithromycin core comprises an azithromycin-containing particle coated with a sustained release coating.

9. A pharmaceutical composition of claim 3 wherein said enteric coating is selected so that the rate of azithromycin ester formation Rₑ in wt %/day at temperature T in ° C. of said pharmaceutical composition is less than or equal to
1.8×10⁸·e^{7070/(T+273)}, and
wherein T ranges from 20° C. to 50° C.

10. The pharmaceutical composition of claim 3 wherein said enteric coating is selected so that the rate of azithromycin ester formation Rₑ in wt %/day at temperature T in ° C. of said pharmaceutical composition is less than or equal to
3.6×10⁷·e^{-7070/(T+273)}, and
wherein T ranges from 20° C. to 50° C.

11. The pharmaceutical composition of claim 3 wherein said enteric coating is selected so that the rate of azithromycin ester formation Rₑ in wt %/day at temperature T in ° C. of said pharmaceutical composition is less than or equal to
1.8×10⁷·e^{-7070/(T+273)}, and
wherein T ranges from 20° C. to 50° C.

12. The pharmaceutical composition of claim 1 wherein said core comprises about 35 to about 55 wt % azithromycin; about 40 to about 65 wt % of a carrier selected from the group consisting of waxes, glycerides, and mixtures thereof; and about 0.1 to about 15 wt % of a dissolution enhancer.

13. The pharmaceutical composition of claim 12 wherein said core comprises about 45 to about 55 wt % azithromycin; about 40 to about 55 wt % of a glyceride, and about 0.1 to about 5 wt % of a poloxamer.

14. The pharmaceutical composition of any of claims 1-4 wherein said multiparticulates further comprise a barrier coat located between said core and said enteric coating; wherein said barrier coat is selected from the group consisting of long-chain alcohols, poloxamers, ethers, ether-substituted cellulosics, sugars, salts, and mixtures thereof.

15. The pharmaceutical composition of claim 20 wherein said enteric coating is a trimellitate-containing coating or a phthalate-containing coating selected from the group consisting of acid phthalates of carbohydrates, amylose acetate phthalate, cellulose acetate phthalate, cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, cellulose acetate trimellitate, and mixtures thereof.
